SEQUENCE LISTING (20 sequences)

SEQ ID NO: 1 - DNA, 24 bp, Artificial
  chemically synthesized primer
tgcatcaccggtcagaaaacaact

SEQ ID NO: 2 - DNA, 24 bp, Artificial
  chemically synthesized primer
ggcccgaaccaggaggaagataaa

SEQ ID NO: 3 - DNA, 21 bp, Artificial
  chemically synthesized primer
cccctcgctctttttatggac

SEQ ID NO: 4 - DNA, 22 bp, Artificial
  chemically synthesized primer
cctggccgctgagtttttgttc

SEQ ID NO: 5 - DNA, 18 bp, Artificial
  chemically synthesized primer
gccccgctttcacctctg

SEQ ID NO: 6 - DNA, 22 bp, Artificial
  chemically synthesized primer
tgccgtttcttgttcttcctct

SEQ ID NO: 7 - DNA, 19 bp, Artificial
  chemically synthesized primer
cagccccgtgttggtaata

SEQ ID NO: 8 - DNA, 19 bp, Artificial
  chemically synthesized primer
cccggaatgaagaatggag

SEQ ID NO: 9 - DNA, 24 bp, Artificial
  chemically synthesized primer
ctatacagcctgcgcgttctcttc

SEQ ID NO: 10 - DNA, 24 bp, Artificial
  chemically synthesized primer
agcttgcgcaggcgggttaggttc

SEQ ID NO: 11 - DNA, 19 bp, Artificial
  chemically synthesized primer
acgcgggccgaggtggaca

SEQ ID NO: 12 - DNA, 20 bp, Artificial
  chemically synthesized primer
gcccccgatgcgggctcaac

SEQ ID NO: 13 - DNA, 20 bp, Artificial
  chemically synthesized primer
accacagtccatgccatcac

SEQ ID NO: 14 - DNA, 20 bp, Artificial
  chemically synthesized primer
tccaccaccctgttgctgta

SEQ ID NO: 15 - DNA, 20 bp, Artificial
  chemically synthesized
tccatgacgttcctgacgtt

SEQ ID NO: 16 - DNA, 20 bp, Artificial
  chemically synthesized
tccatgagcttcctgagctt

SEQ ID NO: 17 - protein, 145 aa, Artificial
  consensus sequence of human, mouse and zebrafish TIR domains
DPLGXTPELFDAFICYCPXDIEFVQEMIRQLEQTDYRLKLCVSDRDVLPGTCVWSIASELIEKRCRRMVVVVSDDYLQSKECDFQTKFALSLSPGAXQKRLIPIKYKAMKKXFPSILRFITXCDYTNPCTKSWFWTRLAKALSLP

SEQ ID NO: 18 - protein, 145 aa, Homo sapiens
DPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 19 - protein, 145 aa, Mus musculus
DPLGQTPELFDAFICYCPNDIEFVQEMIRQLEQTDYRLKLCVSDRDVLPGTCVWSIASELIEKRCRRMVVVVSDDYLQSKECDFQTKFALSLSPGVQQKRLIPIKYKAMKKDFPSILRFITICDYTNPCTKSWFWTRLAKALSLP

SEQ ID NO: 20 - protein, 144 aa, Danio rerio
DPEGTPELFDAFICYCQRDFEFVQEMIRQLEHTDFKLKLCVFDRDVLPGSCVWTITSELIEKRCKRMVVVISDEYLDSEACDFQTKFALSLSPGARNKRLIPVKYKSMSKPFPSILRFLTLCDYTRPCTQAWFWKRLAKALSLP

(19) United States
(12) Patent Application Publication
(10) Pub. No.: US 2009/0220528 A1
(43) Pub. Date: Sep. 3, 2009

(54) Stimulation of Toll-Like Receptors on T Cells

(75) Inventors: Laurence Turka, Merion, PA (US); Andrew E. Gelman, St. Louis, MO (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: THE TRUSTEES OF THE UNIVERISTY OF PENNSYLVANIA CNETER FOR TECHNOLOGY TRANSFER, PHILADELPHIA (PA)

(21) Appl. No.: 11/922,443

(22) PCT Filed: Jun. 15, 2006
(86) PCT No.: PCT/US2006/023341

§ 371 Date: Aug. 4, 2008

Related U.S. Application Data
(60) Provisional application No. 60/691,606, filed on Jun. 17, 2005.

(51) Int. Cl. A61K 39/395; A61K 31/711; A61K 39/00; A61K 35/12; C12N 5/10; C12N 5/06; A61P 37/02

(52) U.S. Cl. 424/173.1; 514/44 A; 424/184.1; 424/93.7; 435/325; 435/375

(57) ABSTRACT

The present invention relates to compositions and methods for modulating Toll-like receptors (TLRs) for enhancing survival of activated CD4+ T cells. The enhanced survival of activated CD4+ T cells provides a means for regulating an immune response.

### STATEMENT REGARDING FEDERALLY SPONSORED RESEARCH OR DEVELOPMENT

This invention was made, in part, using funds obtained from the U.S. Government (National Institutes of Health Grant No. AI41521), and the U.S. Government may therefore have certain rights in this invention.

### BACKGROUND OF THE INVENTION

Toll-like receptors (TLRs) (Kaisho et al., 2001 Trends Immunol. 22:78) mediate the recognition of pathogen-associated molecular patterns (PAMPs) by cells of the innate immune system allowing the detection of infection and inflammation (Medzhitov et al., 1997, Nature 388:394). On (antigen presenting cell) APCs, PAMP engagement of TLRs promotes maturation, a process characterized by the up-regulation of MHC and costimulatory molecules and the secretion of proinflammatory cytokines, which in turn leads to the induction of proliferation and survival pathways in antigen-specific CD4⁺ T cells (Kaisho et al., 2001, Trends Immunol. 22:78). Several distinct molecular pathways contribute to these effects. For example, TCR engagement activates NF-κB, a transcription factor that mediates many inflammatory responses and is important in maintaining activated CD4⁺ T cell survival (Zheng et al., 2003, J. Exp. Med. 197:861). TCR survival signals are further enhanced by costimulation through CD28 that promotes the synthesis of the prosurvival molecule BCl-x_{L} and the cytokine IL-2 (Boise et al., 1995, Immunity 3:87). IL-2 in turn provides survival signals to activated CD4⁺ T cells through induction of Bcl-2 (Mueller et al., 1996 J. Immunol. 156:1764). Furthermore, PAMP-stimulated APCs also secrete type I (interferons) IFNs and IL-15, both of which enhance activated CD4⁺ T cell survival following cessation of APC TCR engagement (Oshiumi et al., 2003, Nat. Immunol. 4:161; Mattei et al., 2001, J. Immunol. 167:1179). Thus, PAMPs clearly promote activated CD4⁺ T cell survival indirectly by initiating maturation responses from APCs.

Interestingly, CD4⁺ T cells also express TLRs suggesting that PAMPs may directly induce activated CD4⁺ T cell survival (Mokuno et al., 2000, J. Immunol. 165:931; Caramalho et al., 2003, J. Exp. Med. 197:403). TLR expression has been reported on γδT cells and regulatory CD4⁺CD25⁺ T cells. However, the function of TLRs on CD4⁺ T cells remains poorly understood. It has recently been reported that stimulation of TLR-4 on regulatory T cells increases the suppressive activity and proliferation of these cells (Caramalho et al., 2003, J. Exp. Med. 197:403). However, whether PAMPs are capable of inducing direct functional responses in activated nonregulatory CD4⁺ T cells or whether TLR-mediated responses in CD4⁺ T cells use the same signaling pathways that have previously been described in APCs is not known.

TLR signaling is initiated through at least two pathways: one dependent on the adaptor molecule myeloid differentiation factor 88 (MyD88) and an other that is MyD88 independent (Takeuchi et al., 2002, Curr. Top. Microbiol. Immunol. 270:155). All TLRs utilize the MyD88 pathway but not all TLRs are dependent on it to mediate all functional responses to PAMPs (Yamamoto et al., 2003, Science 301:640). For example, TLR-4-mediated IL-6 and TNF-αsynthesis by dendritic cells (DCs) is dependent on MyD88, but maturation responses such as costimulatory molecule up-regulation are relatively independent (Kawai et al., 1999, Immunity 11:115; Akira et al., 2000, J. Endotoxin Res. 6:383). In contrast, all TLR-9-mediated functional responses are dependent on MyD88 (Schnare et al., 2000, Curr. Biol. 10:1139). Nevertheless, both pathways lead to the activation of NF-κB and the mitogen-activated protein (MAP) kinases (Akira, 2003 J. Biol. Chem. 278:38105).

In view of the fact that the function of TLRs on CD4+ T cells, particularly nonregulatory CD4+ T cells, remains poorly understood, the present invention serves to provide insight into the role of TLRs on CD4+ T cells. In addition, many methods exist to expand and manipulate this population of cells. However, generation of a large number of these cells have not been successful. Thus there is a need for methods of enhancing the survival of CD4+ T cells both in vitro and in vivo. The present invention satisfies this need.

### BRIEF SUMMARY OF THE INVENTION

The invention includes a composition for increasing T cell proliferation and cytokine production, wherein the composition comprises a Toll-like receptor (TLR) ligand and a T cell stimulator.

In a preferred embodiment, the cytokine produced by the T cell is IL-2 or IL-6.

In one embodiment, the invention includes a TLR ligand that is capable of activating TLR9.

In another embodiment, the invention includes a TLR ligand is capable of activating TLR3.

In yet another embodiment, the TLR ligand is selected from the group consisting of CpG DNA and poly I:C.

In a further embodiment, the TLR ligand is a combination of CpG DNA and poly I:C.

In another embodiment, the T cell stimulator comprises an antibody selected from the group consisting of an anti-CD3 antibody and an anti-CD28 antibody.

In a further embodiment, the T cell stimulator comprises both an anti-CD3 antibody and an anti-CD28 antibody.

The invention also includes a composition for increasing T cell proliferation and cytokine production, wherein the composition comprises a TLR ligand, a T cell stimulator and an antigen having at least one epitope, wherein the epitope is capable of eliciting an immune response in a mammal.

The invention also includes a composition for increasing T cell proliferation and cytokine production, wherein the composition comprises a TLR ligand, a T cell stimulator and a T cell.

In one embodiment, the T cell is an activated T cell. Preferably, the T cell exhibits an enhanced survival characteristic.

The invention also includes a composition comprising one of CpG and poly I:C and a T cell stimulator.

In one embodiment, the T cell stimulator comprises an antibody selected from the group consisting of an anti-CD3 antibody and an anti-CD28 antibody.

In another embodiment, the T cell stimulator comprises both an anti-CD3 antibody and an anti-CD28 antibody.

The invention also includes a T cell that is genetically modified to express elevated levels TLR3 and/or TLR9 compared to an otherwise identical T cell not so modified, wherein contact of TLR3 and/or TLR9 with a TLR ligand enhances the survival of said genetically modified T cell.

In one embodiment, the genetically modified T cell exhibits an enhanced survival characteristic compared to an otherwise identical T cell not so modified. Preferably, the T cell is capable of regulating an immune response.

In a further embodiment, the immune response is associated with a disease selected from the group consisting of an infectious disease, a cancer, and an autoimmune disease.

The invention also includes a method of inducing T cell proliferation and promoting cytokine production, the method comprising activating a T cell with a composition comprising a TLR ligand and a T cell stimulator.

In one embodiment, the T cell proliferation is dependent on NF-κB.

In another embodiment, the method of inducing T cell proliferation and promoting cytokine production is independent of the presence of an antigen presenting cell.

The invention also includes a method of inducing T cell proliferation and promoting cytokine production, the method comprising activating a T cell with a composition comprising one of CpG and poly I:C; and a T cell stimulator.

In one embodiment, the T cell stimulator comprises an antibody selected from the group consisting of an anti-CD3 antibody and an anti-CD28 antibody.

In another embodiment, the T cell stimulator comprises both an anti-CD3 antibody and an anti-CD28 antibody.

In another embodiment, the method of inducing T cell proliferation and promoting cytokine production is independent of the presence of an antigen presenting cell.

In another embodiment, the cytokine is selected from the group consisting of IL-2 and IL-6.

The invention also includes a method of enhancing an immune response in a mammal, the method comprising administering to the mammal a composition comprising a TLR ligand a T cell stimulator.

The invention further provides a method of enhancing an immune response in a mammal, the method comprising administering to the mammal a composition comprising one of CpG and poly I:C; and a T cell stimulator.

The invention also includes a method of enhancing an immune response in a mammal, the method comprising administering to the mammal a T cell that has been stimulated with a composition comprising a TLR ligand and a T cell stimulator.

The invention also provides a method of enhancing an immune response in a mammal, the method comprising administering to the mammal a T cell that has been stimulated with a composition comprising one of CpG and poly I:C; and a T cell stimulator.

Also included in the invention is a method of suppressing an immune response in a mammal, the method comprising administering to the mammal a composition that inhibits and/or reduces expression of a TLR and/or a downstream signaling molecule thereof in a T cell in the mammal. Preferably, the composition is selected from the group consisting of a small interfering RNA (siRNA), an antisense nucleic acid and a ribozyme.

In addition, the invention includes a method of suppressing an immune response in a mammal, the method comprising administering to the mammal a composition that inhibits and/or reduces activity of a TLR and/or a downstream signalling molecule thereof in a T cell in the mammal. Preferably, the composition is selected from the group consisting of a transdominant negative mutant, an intracellular antibody, a peptide and a small molecule.

The invention also includes a method of modulating Foxp3 expression in T cells, the method comprising activating a T cell with a composition comprising one of CpG and poly I:C and a T cell stimulator wherein said stimulator is capable of activating said T cell.

In one embodiment, the composition comprises CpG and Foxp3 expression is reduced.

In another embodiment, the composition comprises poly I:C and Foxp3 expression is induced.

In one aspect, the composition further comprises transforming growth factor beta (TGF-b).

The invention is also directed to the use of a composition comprising a Toll-like receptor (TLR) ligand and a T cell stimulator for preparation of a medicament for: a method of inducing T cell proliferation and promoting cytokine production; a method of enhancing an immune response in a mammal; and a method of modulating Foxp3 expression in a T cell.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting TLR RNA expression patterns in activated CD4⁺CD25⁻ T cells.

FIG. 2 is a chart demonstrating that Poly(I:C) or CpG DNA but not LPS induce NF-κB and MAPK signaling in activated CD4⁺ T cells.

FIG. 3, comprising FIGS. 3A through 3D, is a series of charts demonstrating that Poly(I:C) or CpG DNA directly enhances the survival but not the proliferation of activated CD4⁺ T cells.

FIG. 4, comprising FIGS. 4A through 4D, is a series of charts demonstrating that Poly(I:C) or CpG DNA-mediated survival requires NF-κB activation and is associated with Bcl-x_{L} up-regulation but only CpG DNA-mediated survival is MyD88 dependent.

FIG. 5, comprising FIGS. 5A and 5B, is a chart demonstrating that activated CD4⁺ T cell survival in vivo is enhanced by either poly(I:C) or CpG DNA treatment before adoptive transfer into naive hosts.

FIG. 6 is a chart demonstrating that resting mouse CD4⁺CD25⁻ T cells express TLR9 protein, whereas CD4⁺CD25⁺ Tregs do not.

FIG. 7, comprising FIGS. 7A through 7D, is a series of charts demonstrating that Poly I:C or CpG DNA is able to synergize with T cell stimulation to induce T cell proliferation.

FIG. 8 is a chart demonstrating that Poly I:C or CpG DNA is able to synergize with T cell stimulation in IL-2 protein production.

FIG. 9 is a chart demonstrating that Poly I:C and CpG DNA mediated proliferative responses are TRAF 6 independent.

FIG. 10, comprising FIGS. 10A and 10B, is a series of charts demonstrating CpG DNA stimulated Akt phosphorylation and GSKα phosphorylation in a PI3-kinase dependent manner in CD4⁺ T cells.

FIG. 11 is a chart demonstrating that CpG DNA mediated IL-2 synthesis in CD4⁺ T cells is MyD88 and PI3-kinase dependent.

FIG. 12, comprising FIGS. 12A and 12B, is a series of charts demonstrating that CpG DNA mediated enhancement of CD4⁺ T cell proliferation is MyD88 dependent.

FIG. 13 is a chart demonstrating that MyD88 has a highly conserved putative SH2 binding (YXXM) domain. Majority-SEQ ID NO:17. Human MyD88 TIR-SEQ ID NO:18. Murine MyD88 TIR-SEQ ID NO:19. Zebrafish MyD88 TIR-SEQ ID NO:20.

FIG. 14 is a schematic depicting an experimental model with respect to a chimera with MyD88-deficient T cells to assess the role of MyD88 in T cell responses in vivo.

FIG. 15 is a chart demonstrating that chimeric mice with MyD88-deficient T cells have splenocytes that upregulated CD86 expression in the presence of CpG DNA.

FIG. 16 is a chart demonstrating that chimeric mice with MyD88-deficient T cells have less plasma INF-γ (INF-g) and IL-12 after infection with T gondii.

FIG. 17, comprising FIGS. 17A and 17B, is a schematic depicting a signal transduction pathway involving MyD88 (FIG. 17A). FIG. 17B depicts a strategy for retroviral reconstitution of MyD88−/− CD4⁺ T cells.

FIG. 18 is a chart demonstrating that optimal IL-6 response to LPS or IL-1 is dependent on Y257 residue in a putative SH2 binding sequence in the MyD88 TIR domain.

FIG. 19, comprising FIGS. 19A through 19D, is a series of charts demonstrating that the death domain and residue Y257 of the TIR domain of MyD88 are both required for optimal CpG ODN-induced proliferation of CD4+ T cells.

FIG. 20 is a chart demonstrating that chimeric mice with MyD88-deficient T cells have similar survival to MyD88−/− mice in that both fail to survive the acute phase T. gondii infection.

FIG. 21, comprising FIGS. 21A and 21B, is a series of graphs demonstrating that TLR ligands can modify Foxp3 expression in natural Tregs.

FIG. 22 is a series of graphs demonstrating the effect of TLR ligands on TGF-b induction of Foxp3 expression in adaptive Tregs.

FIG. 23, comprising FIGS. 23A and 23B, is a series of two charts demonstrating that CpG, but not poly I:C, induces IL-6 production in both Th cells and Tregs.

### DETAILED DESCRIPTION

The invention relates to the discovery that activated CD4⁺ T cells or otherwise pre-stimulated T cells express Toll-like receptor (TLR)-3 and TLR-9 but not TLR-2 and TLR-4, and that the treatment of activated CD4⁺ T cells with ligands for TLR-3 and/or TLR-9 promotes T cell survival. In some cases, the T cell survival was observed without augmenting proliferation of the T cell. In addition, the invention relates to the discovery that activation of a TLR on a T cell at the time of T cell stimulation induces a heightened rate of cellular proliferation and promotes enhanced cytokine production. As such, the present invention encompasses compositions and methods for activating a TLR on a T cell prior to, concurrently with, or following stimulation of the T cell.

The present invention includes compositions and methods for activating Toll-like receptors (TLRs) on T cells to induce multiple signalling pathways, to promote T cell proliferation and survival and to promote the development of effector T cell function, including, but not limited to, development of memory T cells. The invention also includes compositions and methods for manipulating TLRs on T cells to modulate an immune response. The invention also includes compositions and methods for modulating Foxp3 expression in T cells. In addition, the present invention includes compositions and methods that can be used to develop active vaccines and adoptive immunotherapy.

The invention is applicable in systems where T cells are expanded ex vivo by stimulation with antibodies to CD3 and/or CD28 in the absence of APCs. However, the invention should not be limited to anti-CD3 and anti-CD28 antibodies for stimulating T cells, but rather any stimulator of T cells can be used. The stimulation of T cells can be additive when a TLR is activated using the methods disclosed herein, such as using agents including, but not limited to, CpG DNA and poly I:C to enhance the survival characteristics of the T cells.

The invention also provides a method of manipulating T cell activation in ex vivo cultures that is not dependent upon the presence of APCs. An application of the present invention includes the areas of immune adjuvants (for vaccines and cancer immunotherapy).

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

“Allogeneic” refers to a graft derived from a different animal of the same species.

“An antigen presenting cell” (APC) is a cell that is capable of activating T cells, and includes, but is not limited to, monocytes/macrophages, B cells and dendritic cells (DCs).

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillian-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “DNA” as used herein is defined as deoxyribonucleic acid.

“Donor antigen” refers to an antigen expressed by the donor tissue to be transplanted into the recipient.

“Recipient antigen” refers to a target for the immune response to the donor antigen.

As used herein, an “effector cell” refers to a cell which mediates an immune response against an antigen. Effector cells include, but are not limited to, T cells and B cells.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “enhanced survival characteristic,” refers to the discovery that following contacting a TLR ligand with a corresponding TLR on a T cell, levels of prosurvival molecules such as BCl-X_{L} are up-regulated compared with a T cell not so contacted.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “modulate” is meant to refer to any change in biological state, i.e. increasing, decreasing, and the like.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “polypeptide” as used herein is defined as a chain of amino acid residues, usually having a defined sequence. As used herein the term polypeptide is mutually inclusive of the terms “peptide” and “protein”.

“Proliferation” is used herein to refer to the reproduction or multiplication of similar forms of entities, for example, proliferation of a cell. That is, proliferation encompasses production of a greater number of cells, and can be measured by, among other things, simply counting the numbers of cells, measuring incorporation of ³H-thymidine into the cell, and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, a “substantially purified” cell is a cell that is essentially free of other cell types. A substantially purified cell also refers to a cell which has been separated from other cell types with which it is normally associated in its naturally-occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

The term “T-cell,” as used herein, is defined as a thymus-derived cell that participates in a variety of cell-mediated immune reactions.

As used herein, a “T cell stimulator,” means an antibody and/or a ligand that, when specifically bound with a cognate binding partner on a T cell, mediates a response by the T cell, including, but not limited to, activation, initiation of an immune response, proliferation, cytokine production and the like. A T cell stimulator can include, but is not limited to, an MHC molecule loaded with a peptide, an anti-CD3 antibody, an anti-CD28 antibody, an antigen and the like.

As used herein, a “therapeutically effective amount” is the amount of a therapeutic composition sufficient to provide a beneficial effect to a mammal to which the composition is administered.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “virus” as used herein is defined as a particle consisting of nucleic acid (RNA or DNA) enclosed in a protein coat, with or without an outer lipid envelope, which is capable of replicating within a whole cell.

### DESCRIPTION

The invention relates to the identification of a novel mechanism by which T cells respond to engagement of TLRs with their respective ligand. The disclosure presented herein demonstrate that activated T cells express TLR-3 and TLR-9 but not TLR-2 and TLR-4. Activation of TLR-3 and/or TLR-9 on T cells directly enhance their survival in a NF-κB dependent manner demonstrating that TLRs on T cells can directly modulate the immune response. In addition, the invention relates to the discovery that activation of a TLR on a T cell at the time of T cell stimulation induces a heightened rate of cellular proliferation and cytokine production.

Based on the present disclosure, T cell development, including but not limited to, proliferation and survival, can be regulated by manipulating a TLR on a T cell. As such, the present invention includes compositions and methods for modulating the expression and/or activity of TLRs on a T cell to regulate survival and proliferation of the cell. The composition of the present invention is useful in providing a therapeutic benefit in cell therapy and/or vaccination.

In an embodiment of the invention, a T cell in which a TLR has been activated exhibits an enhanced survival characteristic compared with an otherwise identical T cell not having the TLR activated. Preferably, the TLR activated is TLR-3 and/or TLR-9.

According to the present invention, a T cell can be expanded in vitro by contacting a TLR with the appropriate TLR ligand on the T cell at the time of T cell stimulation. That is, the invention relates to the discovery that activation of a TLR on a T cell at the time of T cell stimulation induces a heightened rate of cellular proliferation and cytokine production. Preferably, the cytokine is IL-2. In any event, following treatment and culturing of the T cells in vitro according to the methods disclosed herein the T cells are immunologically functional. For example, they are capable of inducing an immune response and therefore can be administered to a patient in need thereof.

In addition to enhancing T cell survival and cytokine production by activating a TLR on the T cell and stimulating the T cell with a T cell stimulator, the present invention also includes compositions and methods for negatively regulating T cell activation. As such, the invention encompasses compositions and methods for suppressing an immune response. As more fully discussed elsewhere herein, one such method is to decrease the expression or inactivate the protein involved in the TLR signaling pathway including, but not limited to, the TLR itself and downstream signaling molecules. One skilled in the art will appreciate, based on the disclosure provided herein, that one way to decrease the mRNA and/or protein levels of a TLR and/or a downstream signaling molecule in a T cell is by reducing or inhibiting expression of the nucleic acid encoding the TLR and/or the downstream signaling molecule. Thus, the protein level of the TLR and/or the downstream signaling molecule in the T cell can also be decreased using a molecule or compound that inhibits or reduces gene expression such as, for example, an antisense molecule, an siRNA or a ribozyme. Alternatively, the activation of the TLR and/or the downstream signaling molecule can be reduced or inhibited by a transdominant negative mutant of the TLR and/or the downstream signaling molecule.

### Regulation of Toll-Like Receptor (TLR)

Based on the disclosure herein, the present invention includes the generic concept for modulating TLR expression and/or activity in a cell. Preferably, the TLR is TLR3 and/or TLR-9. However, the invention should not be construed to only encompass TLR3 and TLR9, but rather include any TLR that is found to induce proliferation of T cells when contacted with its corresponding ligand. Generating a T cell that exhibits an increased expression and/or activity of a TLR provides a means to promote cellular survival and proliferation. As discussed elsewhere herein, cellular survival refers to the fact that following activation of a TLR on a T cell, various signal transduction molecules are activated, such as, but not limited to, BCl-X_{L}, Akt, NF-κB, MyD88, and the like.

With respect to TLR3, it has been demonstrated that activation of TLR3 on a T cell promotes T cell survival. Preferably, activation of the TLR3 with poly I:C induces activation of the T cell in a MyD88-independent manner.

With respect to TLR9, it has been demonstrated that activation of TLR9 on a T cell promotes T cell survival. Preferably, activation of the TLR9 with CpG DNA induces activation of the T cell in a MyD88-dependent manner.

However, while it is thought that the effects of CpG described herein result solely from CpG interaction with TLR9, it is possible that CpG interaction with another TLR or a non-TLR mediated receptor may also contribute to the observed effects of CpG.

Based on the present disclosure activation of TLR3 and/or TLR9 on a T cell induces survival of the T cell without affecting its innate ability to modulate the immune response. Thus, manipulation of a TLR on a T cell, for example activating expression and/or activity of a TLR on a T cell, offers a strategy to induce T cell proliferation and survival thereby inducing an immune response. In addition, activation of a TLR such as TLR3 and/or TLR9 on a T cell promotes cytokine production. Preferably, the cytokine is IL-2.

Expression of a TLR, preferably TLR3 and/or TLR9 can be induced in a cell using a composition comprising an expression vector encoding the TLR. One skilled in the art will appreciate, based on the disclosure provided herein, that one way to increase the mRNA and/or protein levels of a TLR in a cell is by inducing expression of a nucleic acid encoding the desired TLR.

Based on the present disclosure, one skilled in the art will recognize that in addition to being able to activate a T cell and thereby induce a T cell response with respect to activating a TLR on the T cell, the present invention also includes compositions and methods for suppressing a T cell response. In view of the fact that TLRs contribute to survival characteristics and cytokine production in T cells, it can be appreciated that the effects of TLRs on T cell survival can be reduced or inhibited. Such a method can involve decreasing the expression or inactivate the protein involved in the TLR signaling pathway including, but not limited to the TLR itself, and downstream signaling molecules (i.e. PI3-kinase, Akt, GSKα, NF-κB, MyD88 and others). One skilled in the art will appreciate, based on the disclosure provided herein, that one way to decrease the mRNA and/or protein levels of a TLR in a cell is by reducing or inhibiting expression of the nucleic acid encoding the TLR. Thus, the protein level of the TLR and downstream signaling molecules in a cell can also be decreased using a molecule or compound that inhibits or reduces gene expression such as, for example, an antisense molecule, an siRNA or a ribozyme.

An siRNA is an RNA molecule comprising a set of nucleotides that is targeted to a gene or polynucleotide of interest. As used herein, the term “siRNA” encompasses all forms of siRNA including, but not limited to (i) a double stranded RNA polynucleotide, (ii) a single stranded polynucleotide, and (iii) a polynucleotide of either (i) or (ii) wherein such a polynucleotide, has one, two, three, four or more nucleotide alterations or substitutions therein.

Based on the present disclosure, it should be appreciated that the siRNAs of the present invention may effect the target polypeptide expression to different degrees. The siRNAs thus must first be tested for their effectiveness. Selection of siRNAs are made therefrom based on the ability of a given siRNA to interfere with or modulate the expression of the target polypeptide.

In yet another embodiment, the expression of the desired TLR and/or the downstream signaling molecule can be inhibited using an antisense nucleic acid sequence. Preferably, the antisense nucleic acid is expressed by a plasmid vector. The antisense expressing vector is used to transfect a mammalian cell or the mammal itself, thereby causing reduced endogenous expression of the desired TLR and/or the downstream signaling molecule in the cell. However, the invention should not be construed to be limited to inhibiting expression of the desired TLR and/or the downstream signaling molecule by transfection of cells with antisense molecules. Rather, the invention encompasses other methods known in the art for inhibiting expression or activity of a protein in the cell including, but not limited to, the use of a ribozyme.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In another aspect of the invention, the desired TLR and/or the downstream signaling molecule can be inhibited by way of inactivating and/or sequestering the protein. As such, inhibiting the effects of a TLR and/or a downstream signaling molecule can be accomplished by using a transdominant negative mutant. Alternatively an intracellular antibody specific for the desired protein may be used. In one embodiment, the antagonist per se is a protein and/or compound having the desirable property of interacting with a binding partner of the TLR and/or the downstream signaling molecule and thereby competing with the corresponding wild-type protein. In another embodiment, the antagonist is a protein and/or compound having the desirable property of interacting with the TLR and/or the downstream signaling molecule and thereby sequestering the protein. In any event, the TLR and/or the downstream signaling molecule is inhibited and thereby reducing or preventing the normal outcome of activating a TLR and/or a downstream signaling molecule in a T cell.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention for the purpose of regulating expression and/or activity of a TLR.

### Vectors

Whether the purpose is to increase expression or inhibit expression of a mRNA and/or protein level of a desired TLR and/or a downstream signaling molecule, the invention includes an isolated nucleic acid encoding a TLR and/or a downstream signaling molecule, operably linked to a nucleic acid comprising a promoter/regulatory sequence such that the nucleic acid is preferably capable of directing expression of the protein encoded by the nucleic acid. In other related aspects, the invention includes an isolated nucleic acid encoding a TLR and/or a downstream signaling molecule.

The invention encompasses expression vectors and methods for the introduction of exogenous DNA into cells with concomitant expression of the exogenous DNA in the cells. The incorporation of a desired polynucleotide into a vector and the choice of vectors is well-known in the art as described in, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The polynucleotide of the invention can be cloned into a variety of vectors. However, the present invention should not be construed to be limited to any particular vector. Instead, the present invention should be construed to encompass a wide plethora of vectors which are readily available and/or well-known in the art. For example, an the polynucleotide of the invention can be cloned into a vector including, but not limited to a plasmid, a phagemid, a phage derivative, a mammal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the TLR and/or the downstream signaling molecule, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Constitutive promoter sequences may also be used, including, but not limited to the simian virus 40 (SV40) early promoter, immediate early cytomegalovirus (CMV) promoter sequence, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of a TLR and/or a downstream signaling molecule, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the polynucleotide of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

### T Cell Stimulator

A T cell stimulator of the present invention includes an antibody and/or a ligand that when specifically bound with a cognate binding partner on a T cell, mediates a response by the T cell, including, but not limited to, activation, initiation of an immune response, proliferation, cytokine production, and the like. A T cell stimulator can include, but is not limited to, an MHC molecule loaded with a peptide (otherwise known as peptide/MHC tetramer), an anti-CD3 antibody, an anti-CD28 antibody, an antigen and the like.

The present invention includes various methods for stimulating a T cell including, but not limited to, contacting a T cell with whole antigen in the form of a protein, cDNA or mRNA. However, the invention should not be construed to be limited to the specific form of the antigen used for stimulating the T cell. Rather, the invention encompasses other methods known in the art for generating stimulated T cell. Preferably, the T cell is contacted with an anti-CD3 antibody. In another aspect, the T cell is contacted with an anti-CD28 antibody. In yet another aspect, the T cell is contacted with both an anti-CD3 antibody and an anti-CD28 antibody. As discussed elsewhere herein, the T cell can be stimulated prior to, concurrently with, or following activation of a TLR on the T cell.

The invention includes a T cell that has been exposed or otherwise “activated” with a T cell stimulator and activated by the T cell stimulator. For example, a T cell can be activated by contacting with a T cell stimulator before, after or concurrently with contacting TLR with its corresponding ligand on the T cell. A result of such a treatment is the generation of an activated cell exhibiting an enhanced survival characteristic and enhanced cytokine production. In the case where an antigen is used to activate the T cell, the result is an antigen-specific T cell exhibiting an enhanced survival signal and enhanced cytokine production. The T cell may become activated in vitro, e.g., by culture ex vivo in the presence of an antigen, or in vivo by exposure to an antigen.

A skilled artisan would also readily understand that a T cell can be “activated” in a manner that exposes the T cell to a T cell stimulator for a time sufficient to promote activation of signal transduction pathways indicative of T cell activation. For example, a T cell can be exposed to an antigen in a form small peptide fragments, known as antigenic peptides.

The antigen-specific T cell of the invention is produced by exposure of the T cell to an antigen either in vitro or in vivo. In the case where the T cell is contacted with an antigen in vitro, the T cell is plated on a culture dish and exposed to an antigen in a sufficient amount and for a sufficient period of time to allow the antigen to bind to the T cell and induce T cell activation. The amount and time necessary to achieve binding of the antigen to the T cell may be determined by using methods known in the art or otherwise disclosed herein. Other methods known to those of skill in the art, for example immunoassays or binding assays, may be used to detect the presence of antigen on the T cell following exposure to the antigen.

The antigen may be derived from a virus, a fungus, or a bacterium. The antigen may be a self-antigen or an antigen associated with a disease selected from the group consisting of an infectious disease, a cancer, an autoimmune disease.

It is understood that an antigenic composition of the present invention may be made by a method that is well known in the art, including but not limited to chemical synthesis by solid phase synthesis and purification away from the other products of the chemical reactions by HPLC, or production by the expression of a nucleic acid sequence (e.g., a DNA sequence) encoding a peptide or polypeptide comprising an antigen of the present invention in an in vitro translation system or in a living cell. In addition, an antigenic composition can comprise a cellular component isolated from a biological sample. Preferably the antigenic composition is isolated and extensively dialyzed to remove one or more undesired small molecular weight molecules and/or lyophilized for more ready formulation into a desired vehicle. It is further understood that additional amino acids, mutations, chemical modification and such like, if any, that are made in a antigen component will preferably not substantially interfere with the antibody recognition of the epitopic sequence.

### Methods

The invention encompasses a method for inducing proliferation of a T cell. In another embodiment, the invention includes a method for expanding a population of T cells. The T cell so induced or expanded exhibits an enhanced survival characteristic and enhanced cytokine production following treatment of the T cell according to the methods disclosed herein. The method comprises contacting a T cell that is to be expanded with a TLR ligand and a T cell stimulator. As demonstrated elsewhere herein, contacting a T cell with a TLR ligand and a T cell stimulator, stimulates the T cell and induces T cell proliferation such that large numbers of T cells can be readily produced. In the event that an antigen-specific T cell is desired, an antigen can be contacted with a T cell before, concurrently with or after activating a TLR on the T cell. The T cell can be further purified using a wide variety of cell separation and purification techniques, such as those known in the art and/or described elsewhere herein.

The invention encompasses a method for inducing a T cell response to an antigen in a mammal. The method comprises administering a composition comprising a TLR ligand and a T cell stimulator that specifically induces proliferation of a T cell specific for the antigen and induces production of a cytokine. Once sufficient numbers of antigen-specific T cells are obtained using the TLR ligand and T cell stimulator to expand the T cell, the antigen-specific T cells so obtained are administered to the mammal according to the methods disclosed elsewhere herein, thereby inducing a T cell response to the antigen in the mammal. This is because, as demonstrated by the data disclosed herein, that antigen-specific T cells can be readily produced by stimulating resting T cells using the compositions of the invention.

The invention encompasses a method for modulating Foxp3 expression in T cells. Foxp3 expression is a hallmark of regulatory T cells (Tregs). It is thought that Foxp3 functions as a Treg cell lineage specification factor, and is necessary and sufficient for regulatory function in T cells. Modulating Foxp3 expression permits the modulation of the number of Tregs in a T cell population. The number of Tregs can be increased by inducing Foxp3 expression or the number can be decreased by reducing Foxp3 expression. Modulating the number of Tregs may be useful in therapeutic applications. The method comprises activating a T cell with a composition comprising a Toll-like receptor (TLR) ligand and a T cell stimulator. In one embodiment, the TLR ligand is CpG and Foxp3 expression is reduced. In another embodiment, the TLR ligand is poly I:C and Foxp3 expression is induced. The composition for inducing Foxp3 expression optionally further comprises transforming growth factor beta (TGF-b).

### Therapeutic Application

In one embodiment, the invention includes a vaccine. Preferably, the vaccine is a cellular vaccine, whereby a cell may be isolated from a culture, tissue, organ or organism and administered to a mammal in need thereof. The cell may also express one or more additional vaccine components, such as immunomodulators or adjuvants. In a preferred embodiment, the cellular vaccine of the present invention comprises a T cell exhibiting an enhanced survival characteristic and enhanced cytokine production compared to an otherwise identical T cell not treated using the methods of the present invention. The T cell comprising the vaccine has been contacted with a composition comprising a TLR ligand and a T cell stimulator.

In another embodiment, the cellular vaccine comprises a T cell that has been manipulated according to the present invention to acquire increased expression and/or activity of a TLR (i.e. TLR3 and/or TLR9) and/or a downstream signaling molecule. The T cell can also be cultured in vitro in the presence of both a TLR ligand and a T cell stimulator to expand the number of T cells sufficient for therapeutic and/or experimental use. A benefit of generating a T cell that has been activated by contacting with a TLR ligand and a T cell stimulator is that such treatment does not perturb the capacity of the cells to modulate the immune response. Preferably, the treatment of the T cells does not perturb the capacity of the cells to suppress a disease in vivo. Yet the treatment of the cells with a TLR ligand and a T cell stimulator allows for the rapid expansion of T cells. Based on the disclosure herein, T cells treated according to the methods of the present invention exhibit a enhanced survival characteristic. In addition, the T cells following treatment with a TLR ligand and a T cell stimulator exhibit an enhanced cytokine production. Preferably, the T cell exhibits enhanced IL-2 expression.

Following the treatment and culturing of the T cells in vitro, the cells can be administered to a patient in need thereof. Ex vivo procedures are well known in the art and are discussed more fully below. Briefly, cells are isolated from a mammal (preferably a human) and activated in vitro. The cells can also be genetically modified (i.e., transduced or transfected in vitro) with a vector expressing a polynucleotide of the present invention. In any event, the cell can then be administered to a mammalian recipient to provide a therapeutic benefit. The mammalian recipient may be a human and the cell so modified can be autologous with respect to the recipient. Alternatively, the cells can be allogeneic or syngeneic with respect to the recipient.

With respect to administering a T cell of the present invention to a patient in need thereof, a T cell can be manipulated to exhibit an enhanced survival characteristic as well as increased cytokine production using the methods of the present invention. Based on the present invention, TLR activation on a T cell increases the survival characteristic and cytokine production of the cell, but does not perturb the biological function of the T cell. For example, treatment of a T cell according to the present invention does not perturb the capacity of the T cell to induce an immune response in vivo. With respect to manipulation of a T cell to induce expression of a TLR, it is envisioned that such a T cell exhibits an increased survival characteristic and cytokine production following activation of the TLR and stimulation of the T cell with a stimulatory of the present invention.

The procedure for ex vivo expansion of hematopoietic stem and progenitor cells described in U.S. Pat. No. 5,199,942, incorporated herein by reference, can be applied to the cells of the present invention. Other suitable methods are known in the art, therefore the present invention is not limited to any particular method of ex vivo expansion of the cells.

The T cells expanded according to the present invention are administered to a mammal. The amount of cells administered can range from about 1 million cells to about 300 billion. The cells may be infused into the mammal or may be administered by other parenteral means. The mammal is preferably a human patient. The precise dosage administered will vary depending upon any number of factors, including but not limited to, the type of mammal and type of disease state being treated, the age of the mammal and the route of administration.

The cell may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

A T cell (or cells expanded thereof) may be co-administered to the mammal with the various other compounds (cytokines, chemotherapeutic and/or antiviral drugs, among many others). Alternatively, the compound(s) may be administered an hour, a day, a week, a month, or even more, in advance of the T cell (or cells expanded thereby), or any permutation thereof. Further, the compound(s) may be administered an hour, a day, a week, or even more, after administration of T cell (or cells expanded thereby), or any permutation thereof. The frequency and administration regimen will be readily apparent to the skilled artisan and will depend upon any number of factors such as those already discussed elsewhere herein.

In addition to using a cell-based vaccine in terms of ex vivo immunization, the present invention also provides compositions and methods for in vivo immunization to regulate an immune response in a mammal.

With respect to in vivo immunization, the present invention provides a use of a composition for increasing T cell proliferation wherein the composition comprises a TLR ligand and/or a T cell stimulator. As such, a vaccine useful for in vivo immunization comprises at least a TLR ligand and/or a T cell stimulator component. In another aspect, the vaccine further comprises an antigen component, wherein the antigen component is capable of eliciting an immune response in a mammal.

The invention encompasses in vivo immunization for cancer and infectious diseases. In one embodiment, the disorder or disease can be treated by in vivo administration of a TLR ligand and/or a T cell stimulator alone or in combination with an antigen to generate an immune response against the antigen in the patient. Based on the present disclosure, administration of a TLR ligand and/or a T cell stimulator in combination with a antigenic formulation enhances the potency of an otherwise identical vaccination protocol without the use of a TLR ligand and/or a T cell stimulator. Without wishing to be bound by any particular theory, it is believed that immune response to the antigen in the patient depends upon (1) the composition comprising a TLR ligand and/or a T cell stimulator administered, (2) the duration, dose and frequency of administration, (3) the general condition of the patient, and if appropriate (4) the antigenic composition administered.

In one embodiment, the mammal has a type of cancer which expresses a tumor-specific antigen. In accordance with the present invention, an immunostimulatory protein can be made which comprises a tumor-specific antigen sequence component. In such cases, the TLR ligand and/or a T cell stimulator is administered in combination with an immunostimulatory protein to a patient in need thereof, resulting in an improved therapeutic outcome for the patient, evidenced by, e.g., a slowing or diminution of the growth of cancer cells or a solid tumor which expresses the tumor-specific antigen, or a reduction in the total number of cancer cells or total tumor burden.

In a related embodiment, the patient has been diagnosed as having a viral, bacterial, fungal or other type of infection, which is associated with the expression of a particular antigen, e.g., a viral antigen. In accordance with the present invention, an immunostimulatory protein may be made which comprises a sequence component consisting of the antigen, e.g., an HIV-specific antigen. In such cases, a composition comprising a TLR ligand and/or a T cell stimulator is administered in combination with the immunostimulatory protein to the patient in need thereof, resulting in an improved therapeutic outcome for the patient as evidenced by a slowing in the growth of the causative infectious agent within the patient and/or a decrease in, or elimination of, detectable symptoms typically associated with the particular infectious disease.

In either situation, the disorder or disease can be treated by administration of a TLR ligand and/or a T cell stimulator in combination with an antigen to a patient in need thereof. The present invention provides a means to generate a T cell induced immune response to the antigen in the patient. Based on the present disclosure, a skilled artisan would appreciate that a proinflammatory cytokine (i.e. IL-12, TNFα, IFNα, IFNβ, IFNγ and the like) can be added to the treatment regiment disclosed herein to enhance the potency of the composition comprising a TLR ligand and/or a T cell stimulator.

The invention also encompasses the use of pharmaceutical compositions of an appropriate protein or peptide and/or isolated nucleic acid to practice the methods of the invention.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXAMPLES

The invention is now described with reference to the following Examples. These Examples are provided for the purpose of illustration only, and the invention is not limited to these Examples, but rather encompasses all variations which are evident as a result of the teachings provided herein.

### Example 1

### Toll-Like Receptor Ligands Directly Promote Activated CD4⁺ T Cell Survival

TLR engagement by pathogen-associated molecular patterns (PAMPs) is an important mechanism for optimal cellular immune responses. APC TLR engagement indirectly enhances activated CD4⁺ T cell proliferation, differentiation, and survival by promoting the up-regulation of costimulatory molecules and the secretion of proinflammatory cytokines. However, TLRs are also expressed on CD4⁺ T cells, indicating that PAMPs may also act directly on activated CD4⁺ T cells to mediate functional responses. The results disclosed herein demonstrate that activated mouse CD4⁺ T cells express TLR-3 and TLR-9 but not TLR-2 and TLR-4. Treatment of highly purified activated CD4⁺ T cells with the dsRNA synthetic analog poly(I:C) and CpG oligodeoxynucleotides (CpG DNA), respective ligands for TLR-3 and TLR-9, directly enhanced their survival without augmenting proliferation. In contrast, peptidoglycan and LPS, respective ligands for TLR-2 and TLR-4 had no effect. Enhanced survival mediated by either poly(I:C) or CpG DNA required NF-κB activation and was associated with Bcl-x_{L} up-regulation. However, CpG DNA, but not poly(I:C)-mediated effects on activated CD4⁺ T cells required the TLR/IL-1R domain containing adaptor molecule myeloid differentiation factor 88 (MyD88). Collectively, the results disclosed herein demonstrate that PAMPs can directly promote activated CD4⁺ T cell survival, demonstrating that TLRs on T cells can directly modulate adaptive immune responses.

The materials and methods employed in the experiments disclosed herein are now described.

### Mice

BALB/c mice were purchased from The Jackson Laboratory (Bar Harbor, Me.). DO11.10 mice on the BALB/c background have been described previously (Hsieh et al., 1992, Proc. Natl. Acad. Sci. USA 89:6065). MyD88^{-/-} mice have been described previously (Adachi et al., 1998, Immunity 9:143). For these experiments MyD88^{+/-} mice were backcrossed at least five times onto a C57BL/6 background and intercrossed to generate MyD88^{-/-} and MyD88^{+/+} wild-type control littermates.

### CD4⁺ T Cell Purification

In experiments using BALB/c CD4⁺ T cells, splenocytes and lymph node cells were pooled, erythrocyte-depleted by hypotonic lysis, and labeled with CD4-FITC monoclonal antibody (GK1.5; BD Biosciences, Mountain View, Calif.) and CD25-PE monoclonal antibody (PC61; BD Biosciences). Labeled cells were sorted by a FACSVantage high-speed sorter (BD Biosciences) into CD25⁻CD4⁺ populations and then incubated with CD44-biotin monoclonal antibody (IM7; BD Biosciences) and the following mixture of biotinylated monoclonal antibody from the MACS CD4⁺ T cell isolation kit (Miltenyi Biotec, Auburn, Calif.): CD8a (Ly-2), CD11b (Mac-1), CD45R (B220), pan NK (DX5), and Ly-76 (TER-119). These cells were then further incubated with anti-biotin magnetic beads (Miltenyi Biotec) and purified over LS columns (Miltenyi Biotec) in accordance with the manufacturer's recommendations to obtain the naive CD44^{low} CD25⁻CD4⁺ T cell fraction (purity>99%). In experiments using DO11.10, MyD88^{-/-} or MyD88^{+/+} wild-type littermate control mice, CD4⁺ T cells were directly purified from erythrocyte-depleted splenocyte and lymph node cells with the MACS CD4⁺ T cell isolation kit. Purity in these fractions exceeded 96%. The remainder of the cells were CD8⁺ T cells. APC contamination could not be detected by FACS analysis. However, the presence of APCs was routinely assessed by RT-PCR for MHC class II IAβ message. By the limits of detection in the RT-PCR assay, exceeding one APC in 1000 CD4⁺ T cells, the purified CD4⁺ T cell preparations used in all of these studies have <0.1% APC contamination.

### CD4⁺ T Cell Activation

All CD4⁺ T cell activation was conducted in complete culture medium composed of RPMI 1640 (Life Technologies, Grand Island, N.Y.), 1.5 μM 2-ME (Sigma-Aldrich, St. Louis, Mo.), 50 μg/ml gentamicin (Life Technologies), and 10% FCS (Mediatech, Washington, D.C.) at 37° C. in 5% CO₂. Purified CD4⁺ T cells from either BALB/c, MyD88^{-/-}, or MyD88^{+/+} wild-type control littermates were activated on 24-well plates (Costar, Cambridge, Mass.) coated with 1.0 μg/ml CD3ε monoclonal antibody (2C11; BD Biosciences) and 1.0 μg/ml CD28 monoclonal antibody (37.51; BD Biosciences) for 16 hours. In experiments with DO11.10 mice, 1 μg/ml of pOVA, a peptide derived from chicken albumin amino acid residues 322-332 was added to 2×10⁶/ml erythrocyte-depleted splenocyte and lymph node cell pools for 16 hours. Following pOVA-induced activation, CD4⁺ T cell APC complexes were disrupted with 5 mM EDTA/PBS for 10 minutes at 25° C., washed twice in PBS, and purified with magnetic beads using the MACS CD4⁺ T cell isolation kit as described elsewhere herein. Purity of activated DO11.10 CD4⁺ T cells exceeded 96%. As described elsewhere herein, the remainder of the contaminants by FACS analysis were CD8⁺ T cells, with APCs at <0.1% by RT-PCR.

### Semiquantitative RT-PCR

APCs were prepared from BALB/c pooled splenocyte and lymph node cells that were T cell depleted with MACS anti-CD90.2 beads (Miltenyi Biotec). APC, naïve, and activated CD4⁺ T cell total RNA was prepared by lysis with RLT buffer (Qiagen, Valencia, Calif.) and with buffers and columns supplied from the RNAeasykit with DNase I (Qiagen) in accordance with the manufacturer's instructions. RNA was then reversed transcribed using and amplified with the TITANIUM One Step RT-PCR kit (Clontech Laboratories, Palo Alto, Calif.) under nonsaturating conditions. The following PCR cycling conditions were used: one cycle at 95° C. for 3 min followed by 25-28 cycles of 94.5° C. for 30 seconds and 60° C. for 1 minute and a final cycle at 72° C. for 20 minute. Specific primer sequences were as follows: 5′ TLR-2, TGCATCACCGGTCAGAAAACAACT (SEQ ID NO:1); 3′ TLR-2, GGCCCGAACCAGGAGGAAGATAAA (SEQ ID NO:2); 5′ TLR-3, CCCCTCGCTCTTTTTATGGAC (SEQ ID NO:3); 3′ TLR-3, CCTGGCCGCTGAGTTTTTGTTC (SEQ ID NO:4); 5′ TLR-4, GCCCCGCTTTCACCTCTG (SEQ ID NO:5); 3′ TLR-4, TGCCGTTTCTTGTTCTTCCTCT (SEQ ID NO:6); 5′ TLR-5, CAGCCCCGTGTTGGTAATA (SEQ ID NO:7); 3′ TLR-5, CCCGGAATGAAGAATGGAG (SEQ ID NO:8); 5′ TLR-9, CTATACAGCCTGCGCGTT-CTCTTC (SEQ ID NO:9); 3′ TLR-9, AGCTTGCGCAGGCGGGTTAGGTTC (SEQ ID NO:10); 5′ I-Aβ^{d},ACGC-GGGCCGAGGTGGACA (SEQ ID NO:11); 3′ I-Aβ^{d}, GCCCCCGATGCGGGCTCAAC (SEQ ID NO:12); 5′ G3PDH, ACCACAGTCCATGCCATCAC (SEQ ID NO:13); and 3′ G3PDH, TCCACCACCCTGTTGCTGTA (SEQ ID NO:14). PCR products were resolved by 2% agarose gel electrophoresis, stained with ethidium bromide, and imaged with a Gel Doc analyzer (Bio-Rad, Hercules, Calif.).

### TLR Ligand and Inhibitor Reagents

The CpG oligonucleotide TCCATGACGTTCCTGACGTT (SEQ ID NO:15) (CpG DNA) and non-CpG oligonucleotide TCCATGAGCTTCCTGAGCTT (SEQ ID NO:16) (non-CpG DNA) have been described previously (Hemmi et al., 2000, Nature 408:740) and were synthesized on a phosphorothioate backbone and purified by HPLC (Life Technologies). Poly(I:C), poly(C), and poly(dI:dC) were purchased from Amersham Biosciences (Arlington Heights, Ill.) and LPS, derived from the O55:B5 Escherichia coli strain, was purchased from Sigma-Aldrich. PGN was purchased from Invitrogen (Carlsbad, Calif.). TLR ligands used in all experiments were dissolved in PBS except for PGN, which was solubilized in PBS with 0.02% ethanol. SB203580, U0126, NEMO-binding domain peptide (NBD), and NBD-C were all dissolved in DMSO and purchased from Calbiochem (La Jolla, Calif.).

### NF-κB and MAP Kinase Signaling Analysis

BALB/c CD44^{low}CD25⁻CD4⁺ T cells were activated with plate-bound 1.0 μg/ml anti-CD3 and 1.0 μg/ml anti-CD28 monoclonal antibodies for 16 hours, washed, and rested for 8 hours at 37° C. Activated (1.5×10⁶) CD4⁺ T cells were then treated with TLR ligands for the indicated times, lysed in 1×SDS loading buffer (Bio-Rad Life Sciences), resolved on a 12% bis-Tris SDS-PAGE gel (Life Technologies), transferred to nitrocellulose filters (Life Technologies), and either probed with rabbit anti-mouse phospho-specific Abs for p-IκBα, p-p38, p-extracellular signal-regulated kinase (ERK) 1/2, or p-C-Jun N-terminal kinase (JNK)/stress-activated protein kinase (SAPK). To assess total amounts of signaling molecules, filters were also probed with either rabbit anti-mouse IκBα, p-38, ERK-1/2, or JNK/SAPK antibodies. Detection was conducted with HRP-conjugated goat ant-rabbit antibodies, ECL reagent (Amersham), and X-OMAT Film (Kodak, Rochester, N.Y.). All antibodies were purchased from Cell Signal Technologies (Beverly, Mass.).

### Survival and Proliferation Analysis

Following activation, purified CD4⁺ T cells were washed twice in PBS and replated in culture medium at 10⁶/ml. Cultures were left untreated or treated with either TLR ligands and/or inhibitors for indicated times and concentrations. Following incubation, CD4⁺ T cells were washed twice in PBS/2% FBS and stained with CD4-allophycocyanin monoclonal antibody and 7-aminoactinomycin D (7-AAD; BD PharMingen) or annexin (BD PharMingen) and survival was assessed by exclusion of either of these two stains. For absolute live cell counts 50,000 CD45.1⁺ splenocytes labeled with anti-CD45.1-PE monoclonal antibody (A20; BD PharMingen) were also added to stained CD4⁺ T cell sample FACS tubes just before FACS analysis. Live CD4⁺ T cell counts were calculated by taking the ratio of the number of CD4⁺CD45.1⁻7-ADD⁻ events collected to the number CD45.1⁺ events collected and multiplying by 50,000. Proliferation was measured by CFSE dye dilution as previously described (Wells et al., 1997 J. Clin. Invest. 100:3173).

### Adoptive Transfer and Ex Vivo Proliferation Analysis

Five million purified DO11.10 CD4⁺ T cells were activated by pOVA-pulsed APCs (1 μg/ml), purified with magnetic beads, treated with poly(I:C) (90 μg/ml), CpG DNA (30 μM), LPS (100 ng/ml), or left untreated for 16 hours, washed in PBS twice, and then adoptively transferred into BALB/c hosts. At day 30, spleen and peripheral lymph nodes were harvested and stained with anti-CD4 monoclonal antibody and the DO11.10 clonotypic monoclonal antibody KJI-26. Survival of activated DO11.10 CD4⁺ T cells in each host was determined by FACS analysis and is expressed as a percent with respect to the total number of CD4⁺ T cells found in either the host spleen or lymph nodes along with a mean (thick line) for each treatment group. To measure ex vivo proliferative responses, 72-hour quadruplicate cultures were prepared in 96-well plates with 50,000 irradiated T cell-depleted pOVA-pulsed (1 μg/ml) splenocytes and 150,000 CD4⁺ T cells purified with the MACS CD4⁺ T cell isolation kit from day 30 peripheral lymph nodes or spleen pooled from each treatment group. [³H]Thymidine was added to cultures for an additional 8 hour and read on a beta scintillation counter and is expressed as a mean for each treatment group ±SEM.

### Analysis of Antiapoptotic Molecules

For Bcl-2 evaluation, CD4⁺ T cells were permeabilized with 0.1% saponin/0.2% FBS/PBS and stained with anti-Bcl-2 PE monoclonal antibody (3F-11; BD PharMingen) or an isotype hamster IgG-PE control (A19-3; BD PharMingen) and staining was analyzed by FACS. Bcl-x_{L}, Bcl-3, and β-actin were analyzed by Western blotting using rabbit anti-mouse Abs specific for BCl-x_{L} (BD Transduction Laboratories, Lexington, Ky.), Bcl-3 (Santa Cruz Biotechnology, Santa Cruz, Calif.), and β-actin (Accurate Labs, Westbury, N.Y.).

The results of the experiments are now described.

### CD4⁺ T Cells Modulate TLR Expression in Response to TCR Stimulation

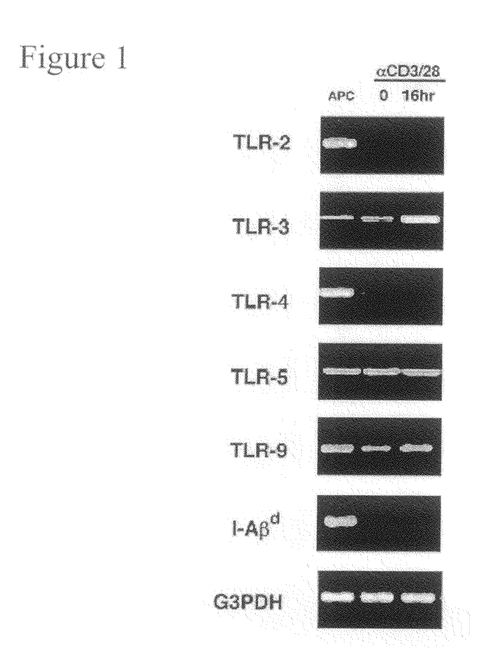

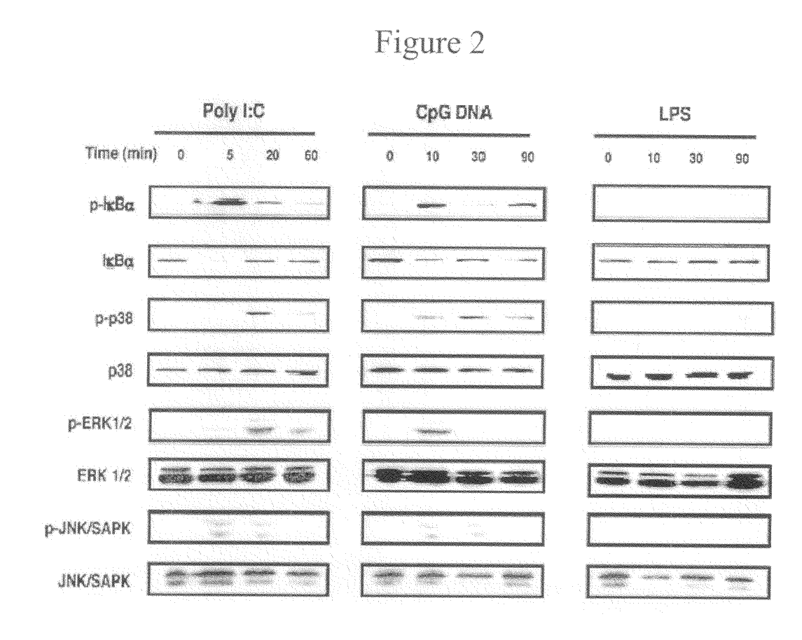

To examine TLR expression patterns in activated CD4⁺ T cells, highly purified naive CD44^{low}CD25⁻CD4⁺ T cells were either left to rest for 8 hours or activated for 16 hours with plate-bound anti-CD3 and anti-CD28 monoclonal antibodies and RT-PCR was performed under nonsaturating conditions for TLRs that are known to have naturally occurring ligands (FIG. 1). TLR-2, -3, -4, -5, and -9 expression was detected in naive CD4⁺ T cells before activation. However, after stimulation, TLR-4 and TLR-2 RNA expression was undetectable while TLR-3 and TLR-9 message was up-regulated. To exclude the possibility that this pattern of TLR expression was partially a result of APC contamination, I-Aβ chain expression was also examined. By the limits of detection of this RT-PCR measurement, which is sensitive to <0.1% APC contamination, no observable I-Aβ chain RNA expression was detected in CD4⁺ T cells before or after activation. Therefore the results disclosed herein demonstrate that CD4⁺ T cells express TLR RNA and modulate its expression following TCR stimulation.

### Poly(I:C) and CpG DNA but not LPS Induce NF-κB and MAP Kinase Activity in Activated CD4⁺ T Cells

TLR ligands induce the activation of nuclear factor NF-κB and MAP kinases in APCs (Akira, 2003, J. Biol. Chem. 278:38105; Martin et al., 2002 Biochem. Biophys. Acta. 1592:265). The next set of experiments were designed to assess whether TLR ligands could also induce NF-κB and MAP kinase activity in activated CD4⁺ T cells (FIG. 2). Both the TLR-3 ligand poly(I:C) and the TLR-9 ligand CpG DNA were able to induce rapid NF-κB activity as evident by phosphorylation of IκBα. In a likewise manner, both ligands were also able to effect phosphorylation of p38 MAP kinase (MAPK), ERK 1/2, and JNK/SAPK. In contrast, LPS did not induce detectable IκBα or MAP family kinase activity consistent with the absence of TLR-4 in activated CD4⁺ T cells. Thus, TLR ligands are able to activate downstream signaling pathways in a manner concordant with the cognate TLR expression pattern in activated CD4⁺ T cells.

### Poly(I:C) or CpG DNA Directly Enhance Activated CD4⁺ T Cell Survival

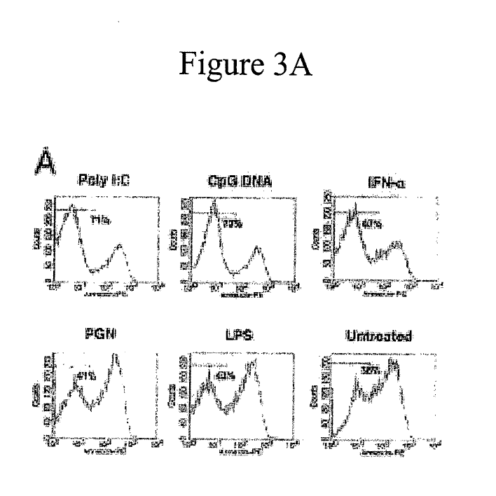

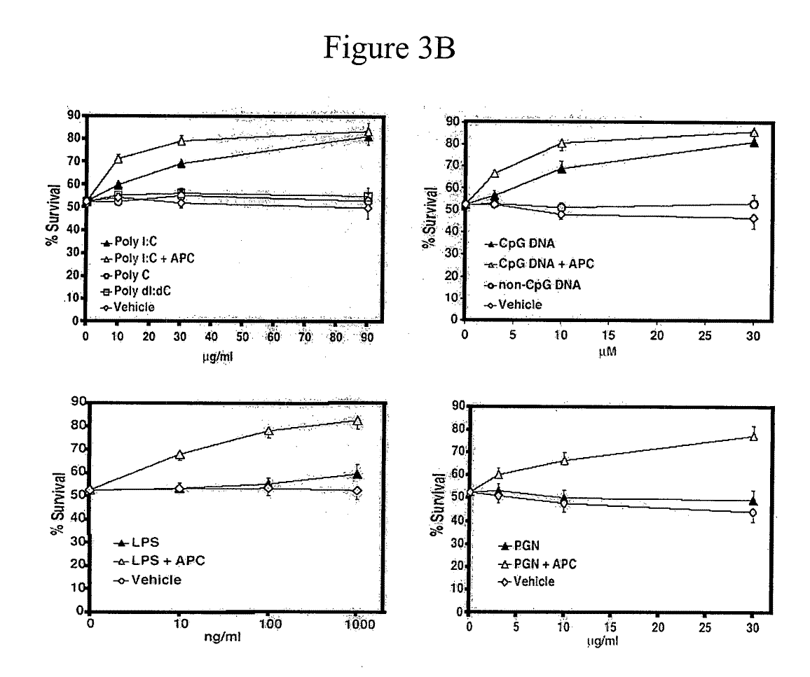

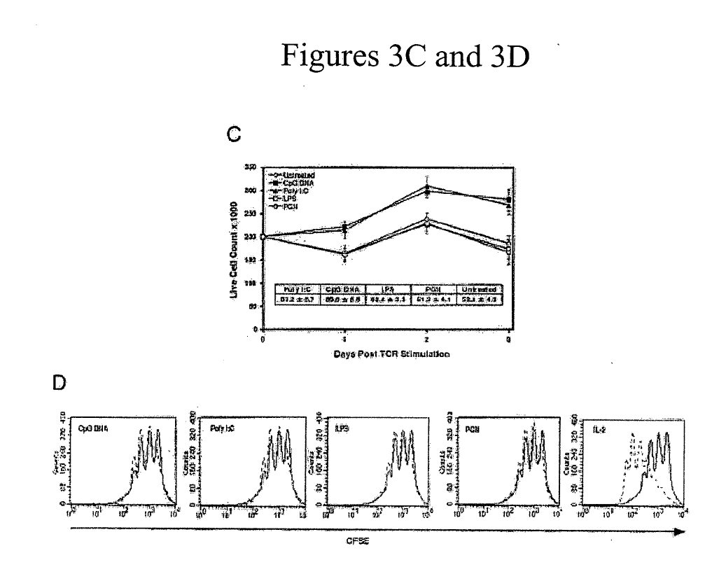

TLR ligands directly promote the survival of several cell types including neutrophils, DC, and B cells (Lundqvist et al., 2002, Cancer Immunol. Immunother. 51:139; Sabroe et al., 2003, J. Immunol. 170:5268; Grillot et al., 1996, J. Exp. Med. 183:381; Grillot et al., 1996, J. Exp. Med. 183:381). Since it was observed that activated CD4⁺ T cells also express TLRs and signal in response to TLR ligands, the next set of experiments were designed to assess whether TLR ligands could directly enhance survival of these cells. DO11.10 CD4⁺ T cells, that encode a transgenic TCR specific for a peptide derived from chicken OVA (pOVA), were activated with pOVA-pulsed APCs for 16 hours ex vivo, purified by magnetic beads to remove all non-CD4⁺ T cells, and replated in unsupplemented culture medium in the absence or presence of TLR ligands. Survival was then assessed 72 hours following activation (FIG. 3A). Poly(I:C) or CpG DNA induced increases of activated CD4⁺ T cell survival from 38% to 71 and 73%, respectively. By contrast, PGN or LPS did not significantly enhance activated CD4⁺ T cell survival promoting only marginal increases from 38% to 41% and 43%, respectively. TLR ligand enhanced mediated survival was comparable to IFN-α, which has been previously reported to enhance the survival for activated T cells (Marrack et al., 1999, J. Exp. Med. 189:521), improving survival from 38% to 67%. It was also observed activated CD4⁺ T cell survival in poly(I:C) and CpG DNA-treated cultures in a dose-dependent manner (FIG. 3B). Enhanced survival was not a nonspecific response to nucleic acids since the addition poly(C), poly(dI:dC), and a control non-CpG DNA had no significant effect. Moreover, LPS and PGN treatment also did not produce significant increments in survival consistent with the absence of detectable TLR-2 and TLR-4 RNA expression on activated CD4⁺ T cells.

To exclude the possibility that poly(I:C) or CpG DNA enhanced survival was an indirect effect arising from contamination with APCs or other non-T cell TLR-bearing cells in the purified CD4⁺ T cell preparations, TLR ligand-treated cultures were also spiked with a T cell-depleted preparation of pooled lymph node cells and splenocytes (APC). The addition of APCs to poly(I:C) and CpG DNA-treated cultures significantly increased dose-responsive survival over TLR ligand-treated cultures alone, suggesting synergy between indirect and direct mechanisms of activated CD4⁺ T cell survival. However, in LPS— or PGN-treated cultures, it was observed that only the addition of APCs could enhance the survival of activated CD4⁺ T cells, thus indicating the functional absence of other TLR-responsive cells in our purified CD4⁺ T cell preparations.

Since percentage survival measurements may not be reflective of viable CD4⁺ T cell numbers in vitro, absolute live cell number counts were quantified for periods up to 72 hours following activation of FACS sorted CD4⁺ T cells with plate-bound anti-CD3 plus anti-CD28 monoclonal antibodies (FIGS. 3C). Consistent with increases in percentage cell survival, concordant increases in live CD4⁺ T cell numbers were observed in poly(I:C) or CpG DNA-treated cultures. By comparison, there were no significant increases in live cell numbers in LPS— or PGN-treated cultures relative to untreated cultures.

Without wishing to be bound by any particular theory, the increases in live cells numbers observed could be explained by TLR ligand-mediated increases in CD4⁺ T cell proliferation. In B cells, LPS, CpG DNA, and poly(I:C) can induce proliferation independently of the B cell receptor (Andersson et al., 1972, Eur. J. Immunol. 2:349; Sun et al., 1997, J. Immunol. 159:3119; Alexopoulou et al., 2001, Nature 413:732). Moreover, LPS has been recently shown to augment regulatory CD4⁺ T cell proliferation (Caramalho et al., 2003, J. Exp. Med. 197:403). To address this issue, CFSE-labeled DO11.10 CD4⁺ T cells were first activated by pOVA-pulsed APCs for 16 hours, purified by magnetic beads, and then treated with TLR ligands and assessed for proliferation 72 hours following activation (FIG. 3D). As expected LPS and PGN treatment, which did not promote direct enhancement of survival of activated CD4⁺ T cells, did not induce more robust proliferative responses in comparison to untreated activated controls. However, and unlike the cytokine IL-2, poly(I:C) and CpG DNA also did not enhance proliferation relative to untreated controls, indicating that the observed increases in viable CD4⁺ T cell numbers were not due to proliferation differences across cultures but solely reflected the enhancement of cell survival.

### Poly(I:C) and CpG DNA-Mediated Survival of Activated CD4⁺ T Cells is Dependent on NF-κB Activation

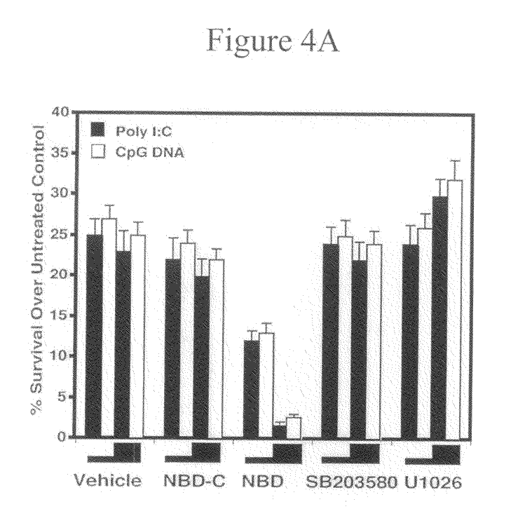

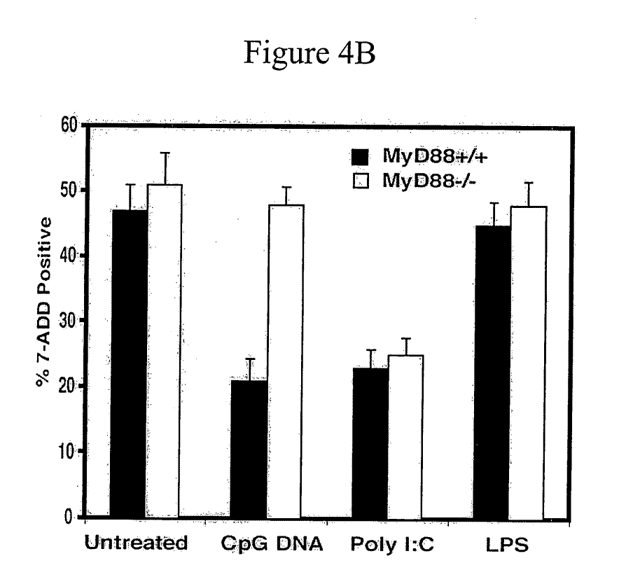

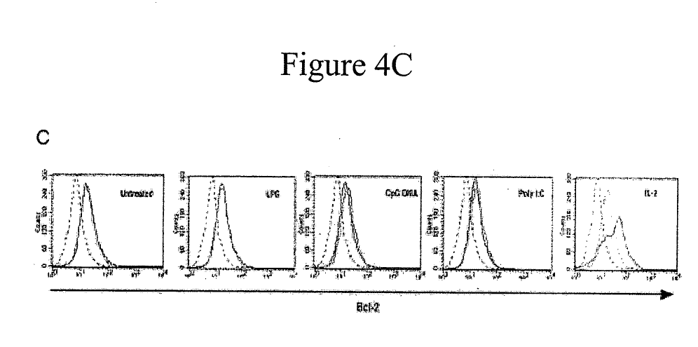

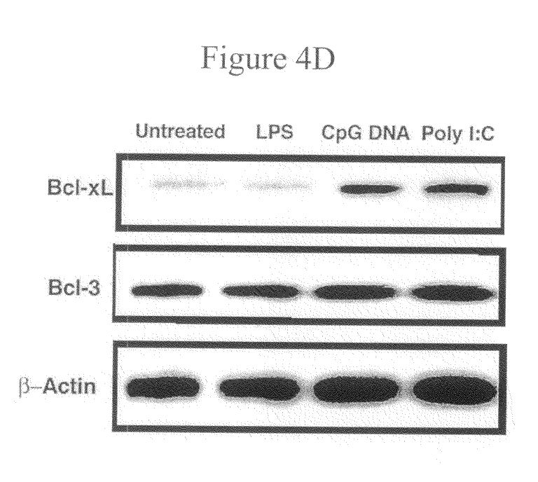

Activation of NF-κB is known to be associated with survival responses in activated CD4⁺ T cells (Zheng et al., 2003, J. Exp. Med. 197:861); Hildeman et al., 2002, Curr. Opin. Immunol. 14:354). To determine whether TLR ligand augmented survival responses in activated CD4⁺ T cells was also dependent on NF-κB activation, IκB phosphorylation was inhibited by using a lipid-soluble peptide (NBD) that selectively binds to the NF-κB essential modifier (NEMO) and blocks its association with the IκB kinases IKKα and IKKβ (IKKαβ) (May et al., 2000 Science 289:1550) (FIG. 4A). NEMO-IKKαβ interaction is necessary for IκB signal-induced phosphorylation and thus inhibiting this association prevents subsequent IκB degradation and NF-κB translocation to the nucleus (Yamaoka et al., 1998, Cell 93:1231). It has been shown that NBD does not modulate JNK activity unlike peptides that directly inhibit NF-κB translocation (May et al., 2000, Science 289:1550). The results presented herein demonstrate that blockade of NF-κB activation by NBD inhibited the ability of both poly(I:C) or CpG DNA to enhance activated CD4⁺ T cell survival. These effects were dose dependent. For example, at 20 μM NBD, TLR ligand augmentation of activated CD4⁺ T cell was substantially reversed. As a control, cultures were treated with a closely related but inactive lipid-soluble form of the peptide NBD-C and observed no significant loss of TLR ligand-mediated survival.

MAPK p38 and ERK 1/2 are also activated by TLR ligands and their function has been shown to be important in controlling T cell-mediated inflammatory responses including survival (Schafer et al., 1999, J. Immunol. 162:659). Therefore, the next set of experiments were designed to assess whether MAPK p38 or ERK 1/2 activation is necessary for TLR ligand-mediated survival in activated CD4⁺ T cells. The ERK1/2 activation inhibitor U0126 or the MAPK p38 inhibitor SB203580 was added to TLR ligand-treated activated CD4⁺ T cells and viability was assessed. It was observed that neither U0126 or SB203580 treatment decreased poly(I:C) and CpG DNA enhanced survival although it was observed that a concentration of 10 μM U0126 induced a small increase in overall survival rates of TLR-treated activated CD4⁺ T cells. Thus, NF-κB but not MAPK p38 or ERK1/2 activation is required to mediate TLR ligand-induced survival of activated CD4⁺ T cells.

CpG DNA but not poly(I:C)-Mediated Survival of Activated CD4⁺ T Cells is Dependent on MyD88

MyD88 is an adaptor molecule recruited to TLRs by TLR ligand engagement and is known to mediate inflammatory responses to many PAMPs (Akira et al., 2003, Biochem. Soc. Trans. 31:637). The absence of MyD88 in APCs makes them completely unresponsive to CpG DNA and is therefore thought to be essential in all TLR-9-mediated responses (Schnare et al., 2000, Curr. Biol. 10:1139). In contrast, deficiency in MyD88 APCs partially eliminates TLR-3-mediated cytokine synthesis but leaves NF-κB, MAPK, and DC maturation responses intact (Alexopoulou et al., 1997, Nature 413:732). Therefore to examine the role of MyD88 in TLR ligand-mediated survival responses in activated CD4⁺ T cells, MyD88^{-/-} activated CD4⁺ T cells were treated with CpG DNA and poly(I:C) and assessed survival (FIG. 4B). It was observed that MyD88 was required to mediate CpG DNA augmented survival of activated CD4⁺ T cells. In contrast, poly(I:C)-enhanced survival responses were left intact in MyD88^{-/-} activated CD4⁺ T cells. Therefore, the results presented herein demonstrate that at least two signaling pathways, MyD88 dependent and MyD88 independent, are capable of mediating direct TLR ligand augmented survival in activated CD4⁺ T cells.

### Poly(I:C) or CpG DNA Treatment of Activated CD4⁺ T Cells Up-Regulates Bcl-x_{L} but not Bcl-2 or Bcl-3

Members of the Bcl family are mediators of activated CD4⁺ T cell survival. Bcl-2 and BCl-x_{L} are both up-regulated in CD4⁺ T cells following antigen priming (Boise et al., 1995, Curr. Opin. Immunol. 7:620). Bcl-3 has been reported to be up-regulated in activated CD4⁺ T cells isolated from adjuvant-treated mice and in overexpression studies has been reported to increase survival (Mitchell et al., 2001, Nat. Immunol. 2:397). Therefore, levels of each of these molecules were measured following TLR ligand treatment of activated CD4⁺ T cells (FIGS. 4C and 4D). Bcl-2 protein levels were not changed by TLR ligand treatment relative to untreated activated CD4⁺ T cell controls. Additionally, Bcl-3 protein levels were also left unaffected despite the fact that all of the TLR ligands used in the experiment are also used as adjuvants (Mitchell et al., 2001, Nat. Immunol. 2:397). However, significant increases in BCl-x_{L} protein in CpG DNA and poly(I:C)— treated activated CD4⁺ T cells was observed over LPS-treated and untreated activated CD4⁺ T cells. Thus, directly mediated activated CD4⁺ T cell survival is associated with specific BCl-x_{L} up-regulation.

Poly(I:C) or CpG DNA Treatment of Activated CD4⁺ T Cells Enhances their Survival In Vivo

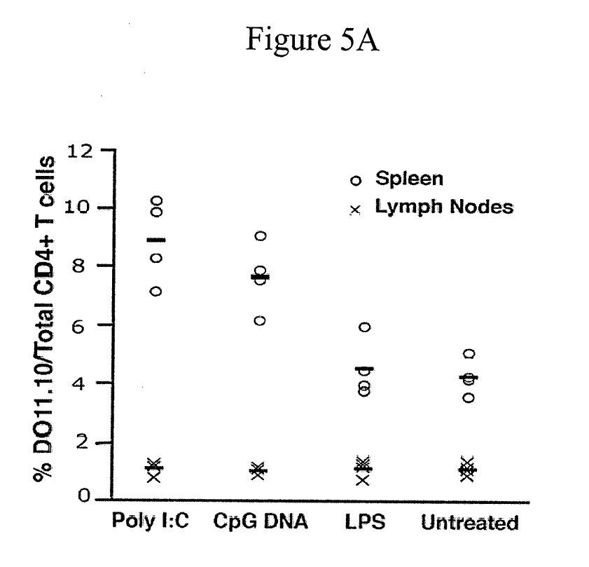

Although TLR ligand-mediated survival of activated CD4⁺ T cells in vitro could be induced, it still remained uncertain whether these cells had a preferential survival advantage in vivo. To address this question, DO11.10 CD4⁺ T cells were first activated with pOVA-pulsed APCs, purified by magnetic beads, treated with TLR ligands for 16 hours, washed, and then adoptively transferred into congenic BALB/c hosts. Survival and ex vivo proliferative responses were assessed 30 days later (FIGS. 5A and 5B). Consistent with what has been previously reported for activated effector CD4⁺ T cells, adoptively transferred activated DO11.10 CD4⁺ T cells seem to preferentially home to the spleen rather than to the peripheral lymph nodes (Bradley et al., 1994, J. Exp. Med. 180:2401). Importantly, poly(I:C) or CpG DNA treatment of activated DO11.10 CD4⁺ T cells increased the percentage of recovered T cells in the host spleen by nearly 2-fold in comparison to spleens from mice that received either LPS-treated or untreated DO11.10 CD4⁺ T cells. Likewise, this 2-fold increase in the percentage of splenic DO11.10 CD4⁺ T cells from hosts that received either poly(I:C)— or CpG DNA-treated activated DO11.10 CD4⁺ T cells was mirrored by a 2-fold increase in the proliferative response to pOVA in comparison to pOVA-induced proliferative responses of splenic CD4⁺ T cells from hosts that received either LPS-treated or untreated activated DO11.10 CD4⁺ T cells. These data suggest that TLR ligands improve recall responses by increasing the number of antigen-specific CD4⁺ T cells in vivo following activation.

TLR message levels in naive CD44^{low}CD25⁻CD4⁺ T cells and activated CD4⁺ T cells was first examined. It was found that activated CD4⁺ T cells, in contrast to naive CD4⁺ T cells, do not express TLR-4 and TLR-2 and increase the expression of TLR-3 and TLR-9 in response to TCR engagement. RNA message levels were used as a proxy for expression due to a lack of antibodies that recognize mouse TLRs. Recent studies have presented an incomplete picture regarding TCR expression in T cells. Nevertheless, both TLR-3 and TLR-9 messages have been found in resting CD4⁺ T cell preparations where naive and activated cells have not been fractionated and in mouse T cell lines (Applequist et al., 2002, Int. Immunol. 14:1065; Zarember et al., 2002, J. Immunol. 168:554; Hornung et al., 2002, J. Immunol. 168:4531). In a single report where the TLR-4 message was specifically assessed in plate-bound anti-CD3 plus anti-CD28 monoclonal antibody-activated regulatory and nonregulatory CD4⁺ T cells, TLR-4 expression was detected in the regulatory but not in the nonregulatory population (Caramalho et al., 2003, J. Exp. Med. 197:403). However, in contrast to the results presented herein and previous studies, TLR-3 and TLR-9 expression was not found on naive CD4⁺ T cells although activated CD4⁺ T cells were not specifically investigated.

In view of the fact that previous studies conducted with TLR ligand-treated APCs from TLR knockout mice demonstrated that NF-κB and MAPK induction requires the expression of cognate TLRs, TLR ligands to validate the observed pattern of TLR expression in activated CD4⁺ T cells (Hemmi et al., 2000, Nature 408:740; Alexopoulou et al., 2001, Nature 413:732; Hoshino et al., 1999, J. Immunol. 162:3749). Poly(I:C) and CpG DNA, but not LPS, induced phosphorylation of I-κB, p38 MAPK, ERK1/2, and JNK/SAPK. Thus, TLR-associated downstream activation pathways are activated by TLR ligands in a manner that matches the observed pattern of TLR expression in activated CD4⁺ T cells.

To further investigate the possible involvement of TLRs in these responses, activated CD4⁺ T cells from mice that are MyD88 deficient were used. The observed requirement for MyD88 to promote CpG DNA-mediated survival in activated CD4⁺ T cells strongly indicates that TLR-9 is mediating these responses since all functional responses mediated by TLR-9 have been reported to be MyD88 dependent (Schnare et al., 2000, Curr. Biol. 10:1139). In contrast, most TLR-3-mediated poly(I:C) responses are MyD88 independent including NF-κB activation (Alexopoulou et al., 2001, Nature 413:732). Poly(I:C) can also directly activate two intracellular pattern recognition receptors, dsRNA-dependent protein kinase (PKR) and 2′-oligoadenylate synthetase/RNase L (Diaz-Guerra et al., 1997, Virology 236:354; Gil et al., 1999, Mol. Cell. Biol. 19:4653). Both of these factors when functioning coordinately in virally infected cells inhibit protein translation leading to apoptosis, thus making them unlikely targets to mediate poly(I:C)-induced survival. However, intracellular PKR activation does induce NF-κB and MAPK responses, which provides the possibility that survival responses may be initiated by this manner (Iordanov et al., 2001, Mol. Cell. Biol. 21:61). The results presented herein argue that this is quite unlikely since intracellular poly(I:C) PKR-mediated TLR-3-independent responses requires liposomal encapsulation of poly(I:C) (Diebold et al., 2003, Nature 424:324). If PKR activation does play a role in survival, it may be via an indirect mechanism through its recruitment to the TLR-3 proximal signaling complex following poly(I:C) stimulation (Jiang et al., 2003, J. Biol. Chem. 278:16713).

Since TLR ligand-mediated survival in several cell types is NF-κB dependent, it was assessed whether the same were true in activated CD4⁺ T cells. It was chosen to inhibit NF-κB activation with NBD, a peptide that prevents IκB phosphorylation through selectively preventing the association of IKKαβ with its regulatory protein NEMO. In the MyD88-dependent TLR signaling pathway, IKKαβ activation has been shown to be requisite for IκB phosphorylation (Wang et al., 2001, Infect. Immun. 69:2270). Moreover, LPS-mediated B cell survival requires the presence of IKKβ and IKKα (Li et al., 2003, J. Immunol. 170:4630; Kaisho et al., 2001, J. Exp. Med. 193:417). For some functional responses, MyD88-independent IκB phosphorylation may be controlled by two other IKK homologues, IKKε and TANK-binding kinase 1 (TBK-1), both of which have been shown to control poly(I:C)-induced IFN-β synthesis (Tojima et al., 2000, Nature 404:778; Fitzgerald et al., 2003, Nat. Immunol. 4:491). The observations of MyD88-independent poly(I:C)-mediated survival in activated CD4⁺ T cells raises the question of whether IKKκ and TBK-1 could also be playing a role in mediating survival responses. Without wishing to be bound by any particular theory, it is believed this is not likely, since NBD which blocks IKKαβ, but not IKKε/TBK-1 activity, was able to inhibit poly(I:C)-mediated survival of activated CD4⁺ T cells. Moreover, poly(I:C) signaling through TLR-3 has been previously reported to activate IKKαβ, and catalytically inactive IKKβ mutants inhibit poly(I:C)-mediated NF-κB-dependent transcription (Gil et al., 2001, Oncogene 20:385; Mitchell et al., 2002, Ann. NY Acad. Sci. 975:114). Thus, the data in activated CD4⁺ T cells indicates that the IKKαβ/NEMO complex promotes TLR ligand-mediated NF-κB activation to enhance survival. In contrast, the results herein present evidence that MAPK p38 or ERK 1/2 activation is not necessary for TLR ligand-activated CD4⁺ T cell survival.

The effects of TLR ligands on the expression levels of prosurvival molecules was also examined. Recognizing studies that suggest that PAMP-mediated survival may be dependent on Bcl-3 levels (Mitchell et al., 2001, Nat. Immunol. 2:397), levels of this molecule in TLR ligand-treated activated CD4⁺ T cells was first assessed. Significant differences in Bcl-3 expression in poly(I:C) or CpG DNA-treated activated CD4⁺ T cells was not observed when compared with untreated activated CD4⁺ T cell controls. These observations may be explained by the dependence on CD40 costimulation to promote Bcl-3 up-regulation in activated CD4⁺ T cells in these previous studies (Mitchell et al., 2002, Ann. NY Acad. Sci. 975:114). Bcl-2 levels and BCl-x_{L} levels were also examined in TLR ligand-treated activated CD4⁺ T cells and it was found that BCl-x_{L} but not Bcl-2 is up-regulated following TLR ligand treatment. This result is in agreement with previous work on PAMP-stimulated B cells and DCs (Lundqvist et al., 2002, Cancer Immunol. Immunother. 51:139; Grillot et al., 1996, J. Exp. Med. 183:381). Without wishing to be bound by any particular theory, since the BCl-x_{L} gene is known to be a downstream target of NF-κB, it is believed that BCl-x_{L} is promoting TLR ligand-mediated survival (Caamano et al., 2002, Clin. Microbiol. Rev. 15:414).

In conclusion, the results presented herein provide evidence that TLR ligands directly enhance the survival of activated CD4⁺ T cells. TLR-mediated survival had the net effect of increasing expansion and slowing contraction rates of activated CD4⁺ T cells without accentuating proliferation. It has been hypothesized that adjuvant-induced activated CD4⁺ T survival can be mediated through APCs by the secretion of proinflammatory cytokines (Hildeman et al., 2002, Curr. Opin. Immunol. 14:354). Interestingly, the results presented herein indicate that for two such PAMPs that are effective adjuvants, poly(I:C) and CpG DNA, adjuvant-mediated survival of activated CD4⁺ T cells may not require APCs. Moreover, in contrast to the indirect means by which TLR ligands control CD4⁺ T cell responses through APCs, direct effects may allow antigen-specific CD4⁺ T cells to respond to PAMPs in situations where APC function is suboptimal, perhaps due to infection (Arrode et al., 2003, Curr. Top. Microbiol. Immunol. 276:277). For example, some viruses which use dsRNA intermediates in their own life cycle, also encode products that inhibit DC maturation and cytokine synthesis and thereby promote infection by attenuating appropriate CD4⁺ T cell responses (Jude et al., 2003, Nat. Immunol. 4:573; Engelmayer et al., 1999, J. Immunol. 163:6762). This effect could be counteracted by augmented CD4⁺ T cell survival responses driven by the release of PAMPs such as dsRNA. Thus, like cells in the innate immune system, activated CD4⁺ T cells may also retain the capability to sense the inflammatory environment by directly responding to PAMPs. This may represent a novel mechanism by which PAMPs promote adaptive immune responses.

### Example 2

### Effects of TLR Ligation at the Time of T Cell Stimulation

Mammalian TLRs are a highly conserved family of molecules which have been known to have key functions in the innate immune system. There are at present eleven known TLRs. Their extracellular domains bind what have been termed PAMPs such as LPS, double stranded RNA, flagellin, CpG DNA, and the like. PAMPs have three key features—they are found only on pathogenic organisms and not on host cells, they are invariant within a class of organisms, and they are required for pathogen survival (i.e., escape mutants do not exist).

Until recently, the primary known role for TLRs has been to activate cells of the innate immune system, such as macrophages and dendritic cells (antigen presenting cells—APCs), thus providing an early warning and mechanism of defense until the adaptive immune system (T and B cells) is able to respond. TLR stimulation of APCs induces the expression of MHC class II, costimulatory ligands such as CD80 and CD86, and the secretion of inflammatory cytokines such as IL-6, IL-12, IFN-α and IFN-β.

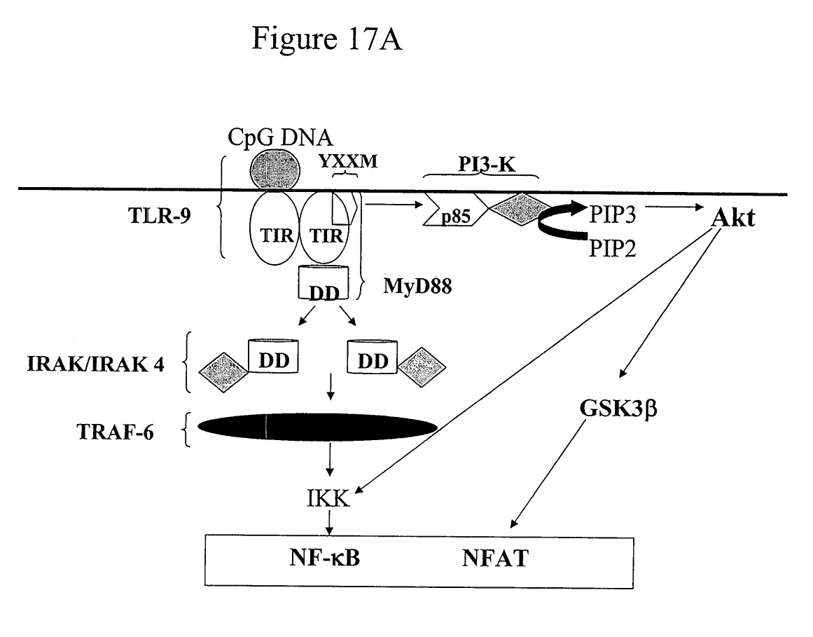

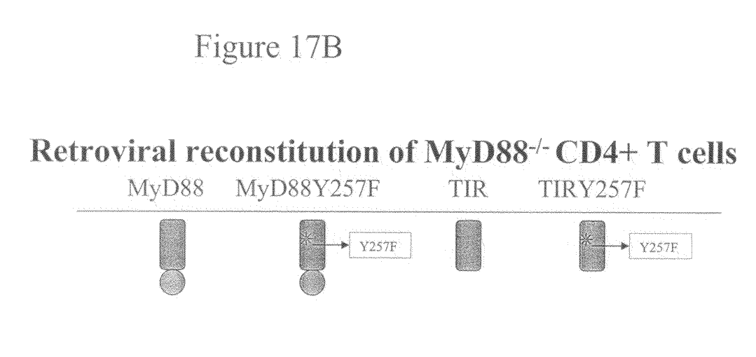

Recently it has been reported that multiple cell types other than innate immune cells also express TLRs. As discussed elsewhere herein, T cells express TLRs -3, -5, and -9, and ligation of either TLR3 or TLR9 on pre-stimulated T cells induced multiple signaling pathways, including NF-κB activation. Further, it has been demonstrated that TLR ligation promoted T cell survival in vitro, an effect which was dependent upon NF-κB. It was observed that augmentation of survival by poly I:C (a TLR3 ligand) was MyD88-independent, while augmentation by CpG DNA (a TLR9 ligand) was MyD88 dependent. This is consistent with the known requirement of MyD88 for TLR9 signaling, and the lack of use of MyD88 by TLR3. FIG. 17A depicts a schematic representation of a signal transduction pathway involving MyD88.

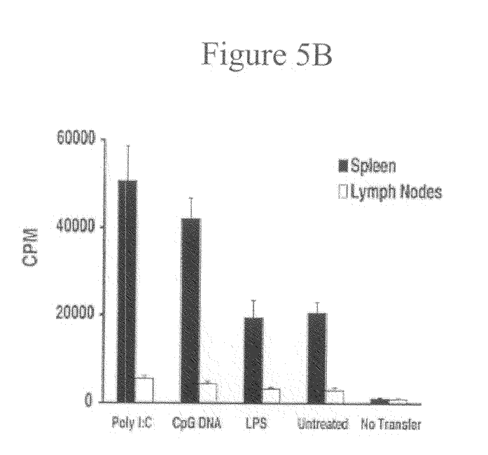

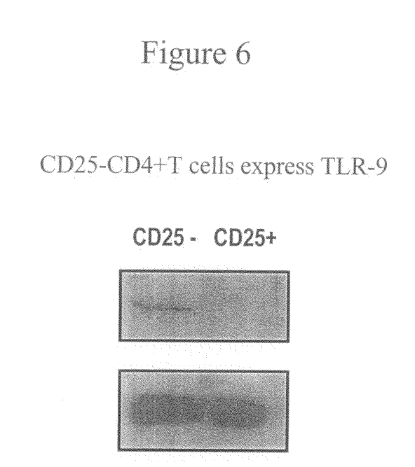

The present experiments were designed to assess the effects of TLR ligation at the time of T cell stimulation, and analysis of signaling pathways which mediate them. As an initial matter, it was demonstrated that that resting mouse CD4+CD25− (i.e., non-regulatory) T cells expressed TLR9 protein, whereas CD4⁺CD25+ Tregs, otherwise known as regulatory T cells, do not (FIG. 6).

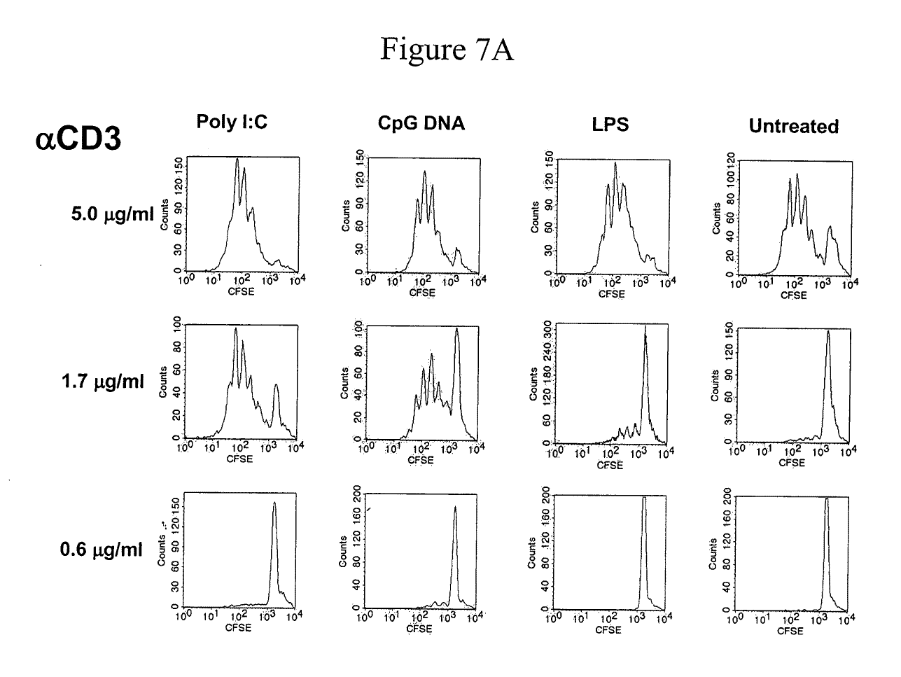

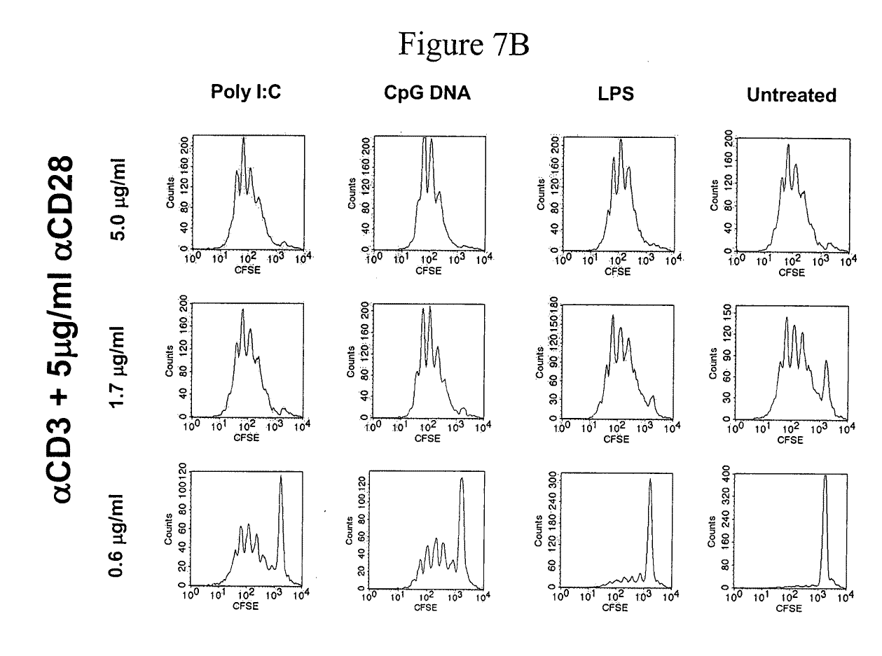

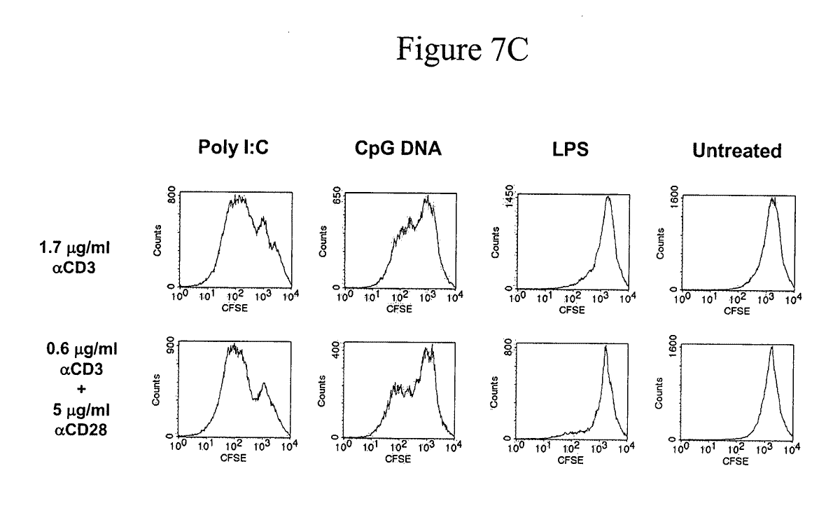

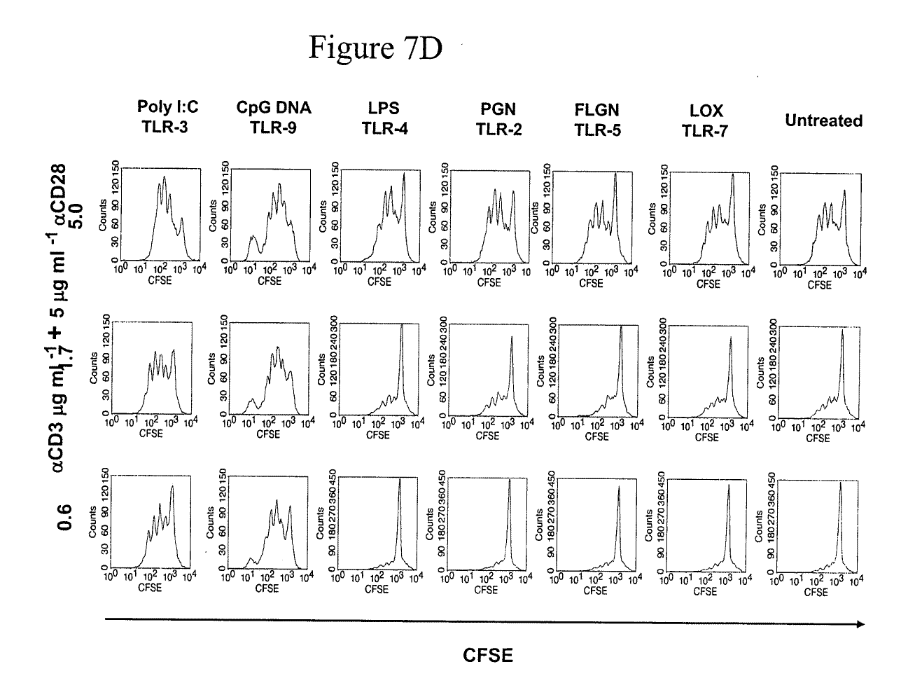

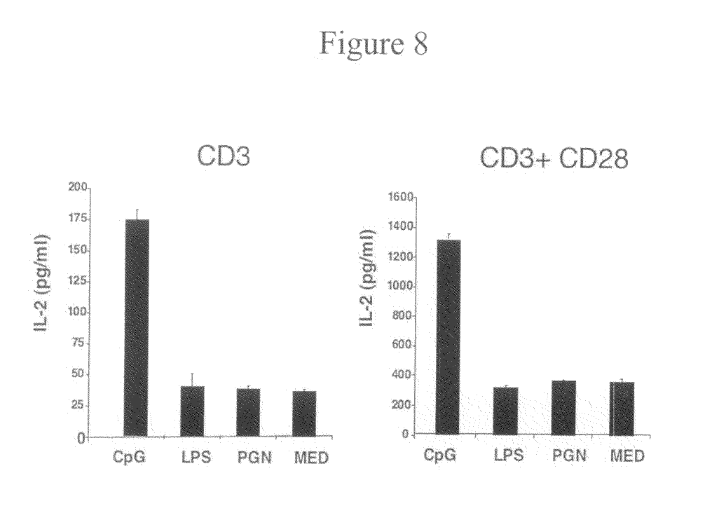

It was also observed that TLR9 stimulation of polyclonal T cells (using CpG DNA, a TLR9 ligand) synergized with sub-mitogenic concentrations of anti-CD3 monoclonal antibody to induce vigorous proliferation (FIG. 7A). Similar results were observed when anti-CD28 was used in combination with anti-CD3 (FIGS. 7B-7D). IL-2 production from treat T cells were also measured (FIG. 8). These effects were additive to those of anti-CD28 stimulation.

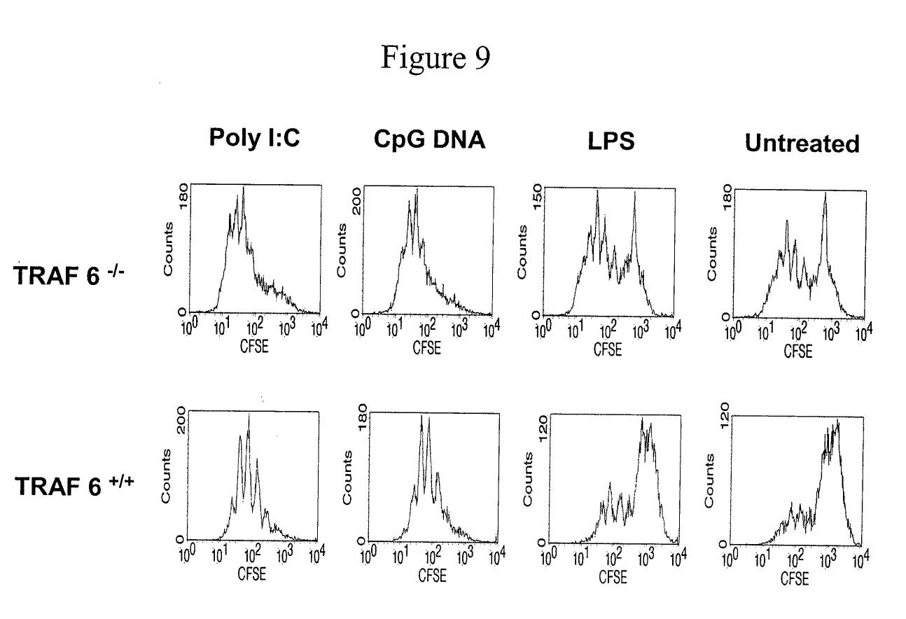

It was also observed that the effects of CpG DNA and poly I:C did not require TRAF6, an adaptor molecule which is believed to couple TLR9 to NF-κB (FIG. 9). This led to the examination of other signaling pathways downstream of TLR9. For example, experiments were designed to assess the role of phosphatidyl inositol 3 kinase (PI3K) in TLR9 activation because of the ability of TLR ligation to synergize with TCR stimulation for IL-2 production and proliferation was reminiscent of CD28 functions. PI3K is a lipid kinase which catalyzes the phosphorylation of phosphatidyl inositol bis 4, 5, phosphate (PIP2) into phosphatidyl inositol tris 3, 4, 5, phosphate (PIP3). PIP3 is an important cell signaling molecule, activating protein kinase B/Akt, and other downstream pathways. In T cells, PI3K activation is a major known downstream signaling pathway of the T cell costimulatory receptor CD28.

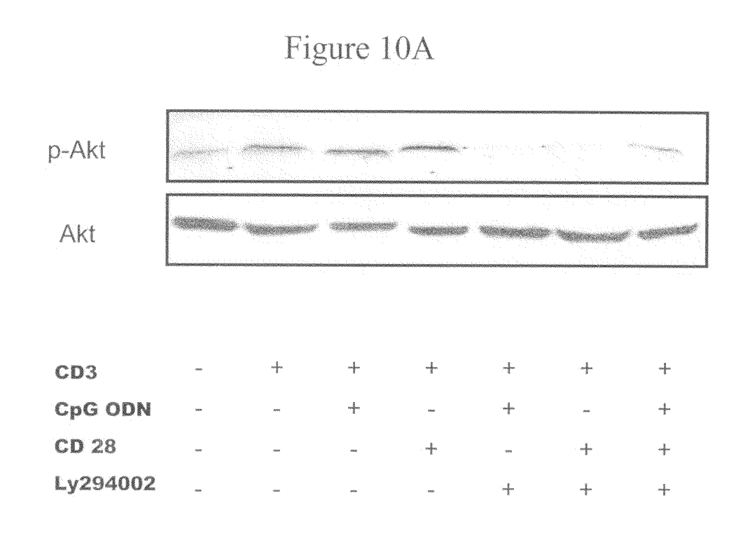

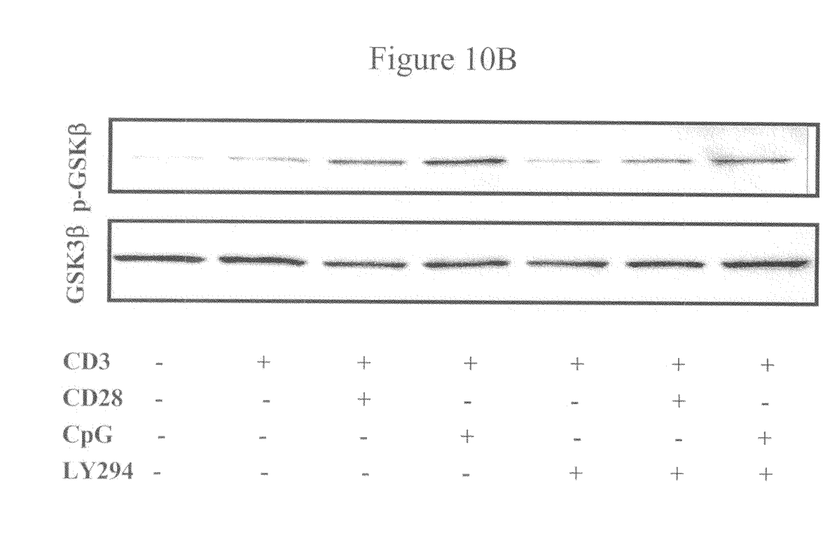

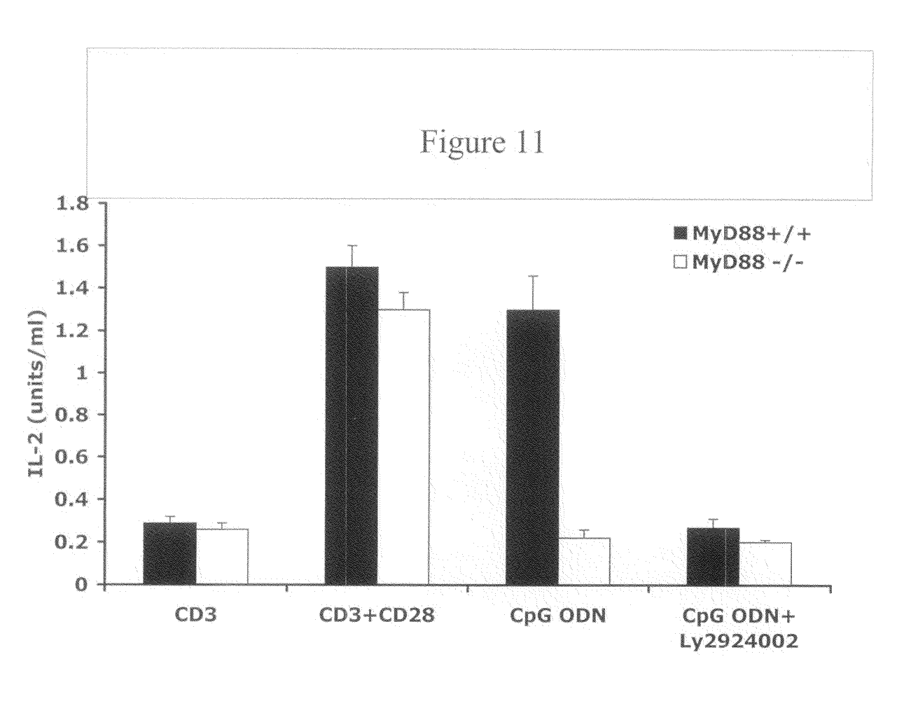

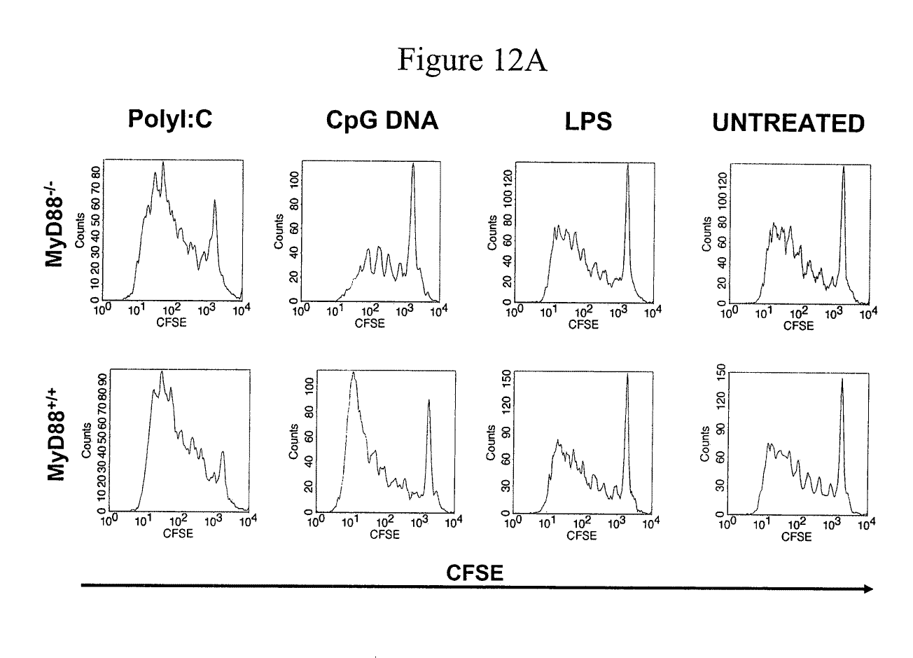

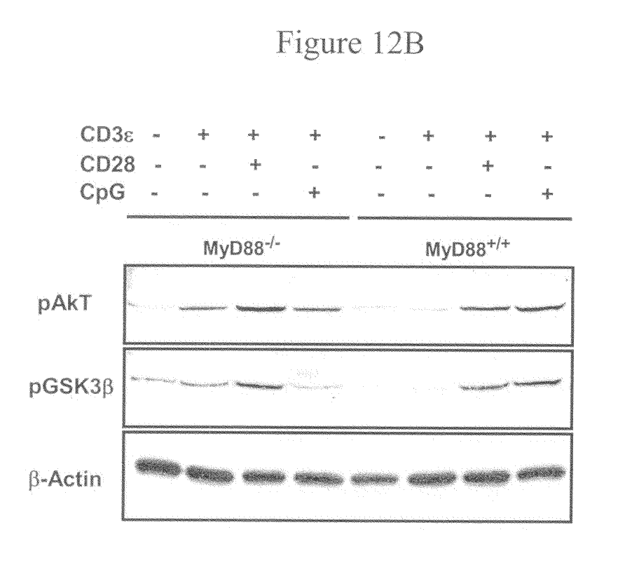

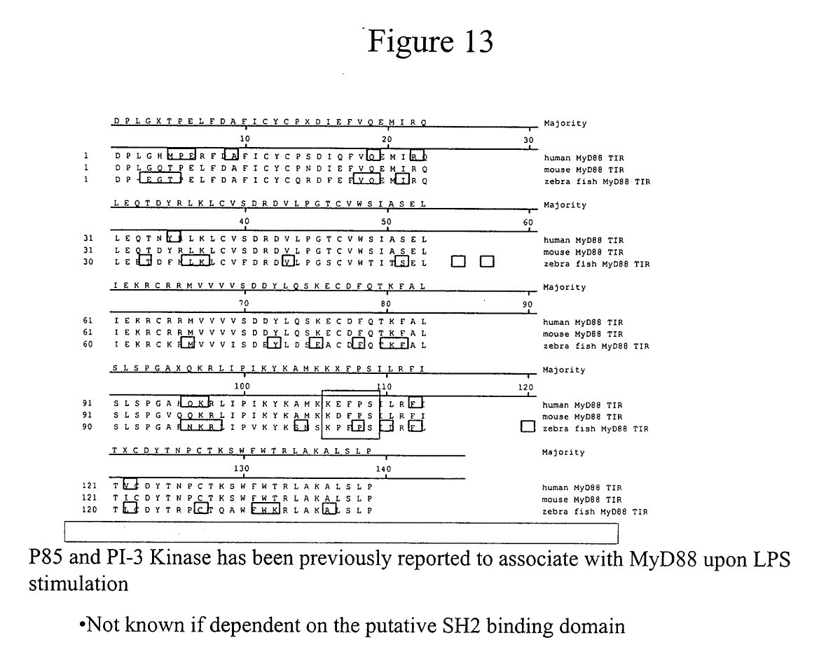

It was observed that CpG DNA activates the PI3K pathway, as indicated by the expression of phosphorylated Akt (FIG. 10A) and phosphorylated GSK3β (FIG. 10B). CpG-mediated augmentation of IL-2 production and proliferation requires MyD88 and is blocked by PI3K inhibition (FIGS. 11 and 12). Sequence analysis shows a potential PI3K binding site in MyD88 and therefore experiments can be designed by making appropriate mutants for expression in MyD88 deficient T cells to determine the role of this motif (FIG. 13).

### Example 3

### In Vivo Effect of TLR Signals on T Cells

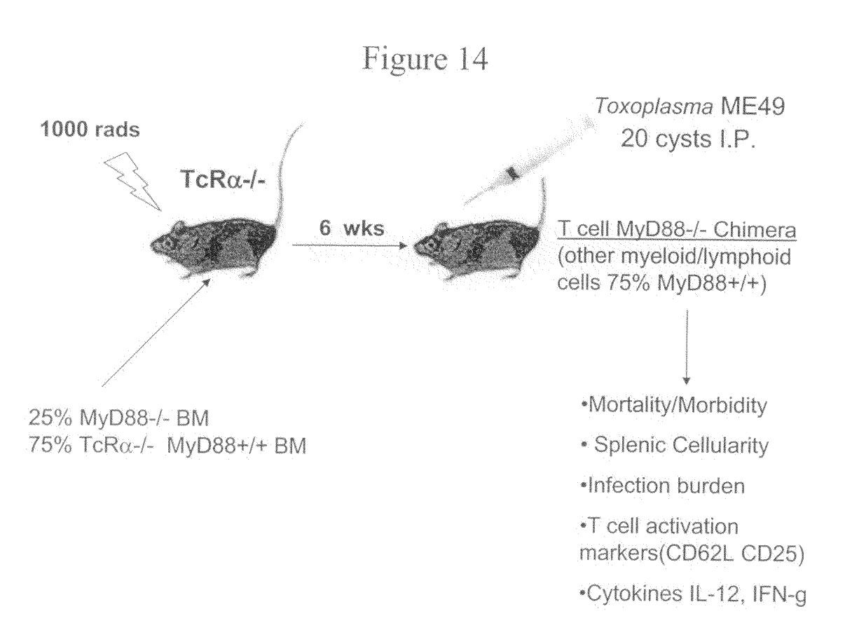

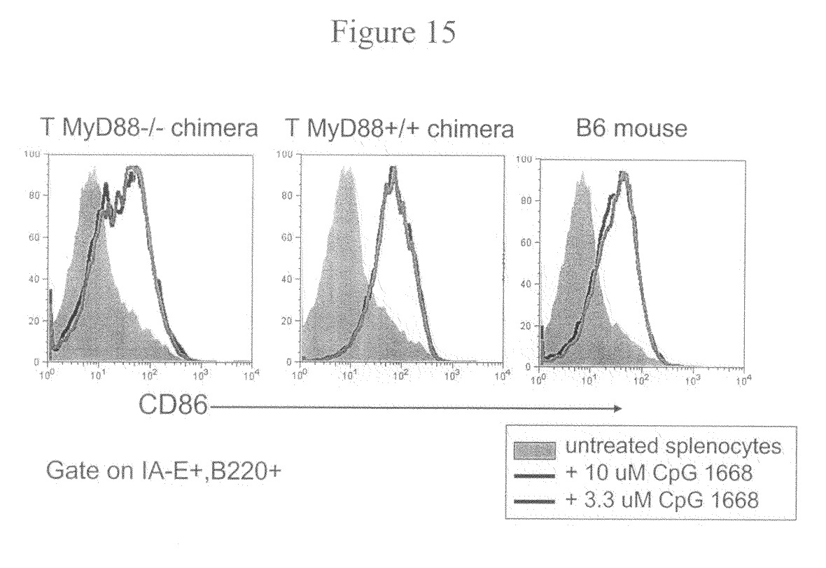

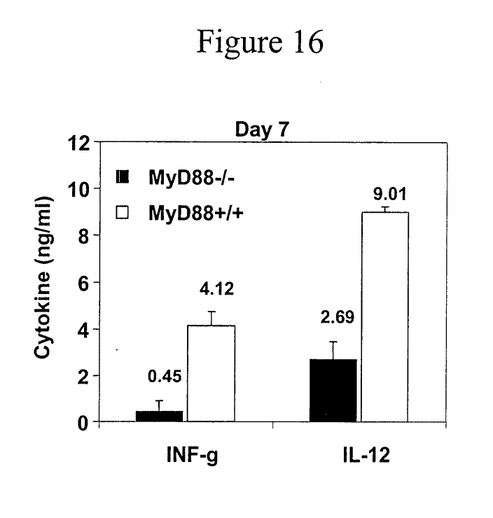

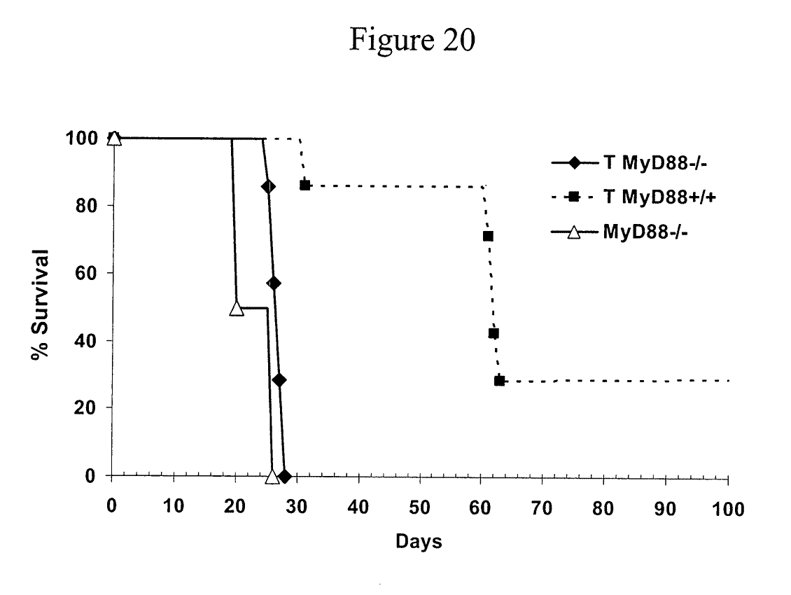

To isolate TLR signaling deficiency to T cells, MyD88-deficient mice was used because MyD88 has been shown to be required for TLR-mediated effects, except those through TLR3 and a subset of those through TLR4 (MyD88 is also used for the IL-1R and IL-18R). The experimental model is depicted on FIG. 14. The experimental model is based on the reported finding that MyD88-deficient mice die rapidly following T. gondii infection, and the ability to make chimeric animals in which the MyD88 deficiency is functionally restricted to T cells. Without wishing to be bound by any particular theory, although some of the APCs in the chimeric mice are MyD88 deficient, it is believed they are a small minority, and the results depicted in FIG. 15 demonstrate that the response of APCs in the chimeras to the MyD88-dependent stimulus CpG DNA is essentially normal. Despite these normal APC responses, the data depicted in Table 1 and FIG. 20 demonstrates that mice lacking MyD88 on T cells are essentially as sensitive to T. gondii as are complete MyD88 knockouts. This is associated with decreased early (day 7) serum levels of both IFN-γ and IL-12 (FIG. 16). The latter in particular is surprising since IL-12 is an APC product and suggests a positive feedback loop involving IFN-γ and IL-12.

Further, it has been observed that chimeric mice with MyD88-deficient T cells have similar survival to MyD88−/− mice in that both fail to survive the acute phase T. gondii infection.

TABLE 1

| | | (Expected cells |
|---|---|---|
| Mouse | Survival (days) | missing MyD88) |
| MyD88−/− mouse | 20, 26 | all |
| Chimera, TCRα−/− and | 31, 61, 62, 62, 63, | None |
| MyD88+/+ marrow | 75+, 75+ | |
| Chimera, TCRα−/− and | 25, 26, 26, 27, 27, | all T cells, |
| MyD88−/− marrow | 28, 28 | minority of APCs |

Experiments were designed to investigate the signaling pathways downstream of TLRs in T cells to determine how activation of PI3K and NF-κB “map” to specific observed effects. Experiments have also been designed to determine whether TLR ligation can prevent anergy induction in T cells. Further, MyD88−/− mice can be crossed onto a TCR transgenic background to determine how antigen-specific responses in vivo are influenced by TLR signaling pathways on T cells.

Without wishing to be bound by any particular theory, it is believed that the requirement for MyD88 in the T. gondii system is due to the role of MyD88 in TLR signaling and not in IL-1R or IL-18R signaling. This is based on studies using blocking reagents and/or knockout animals which showed no requirement for IL-1 or IL-18 in the response to T. gondii.

### Example 4

### Expression and Function of TLRs on T Cells

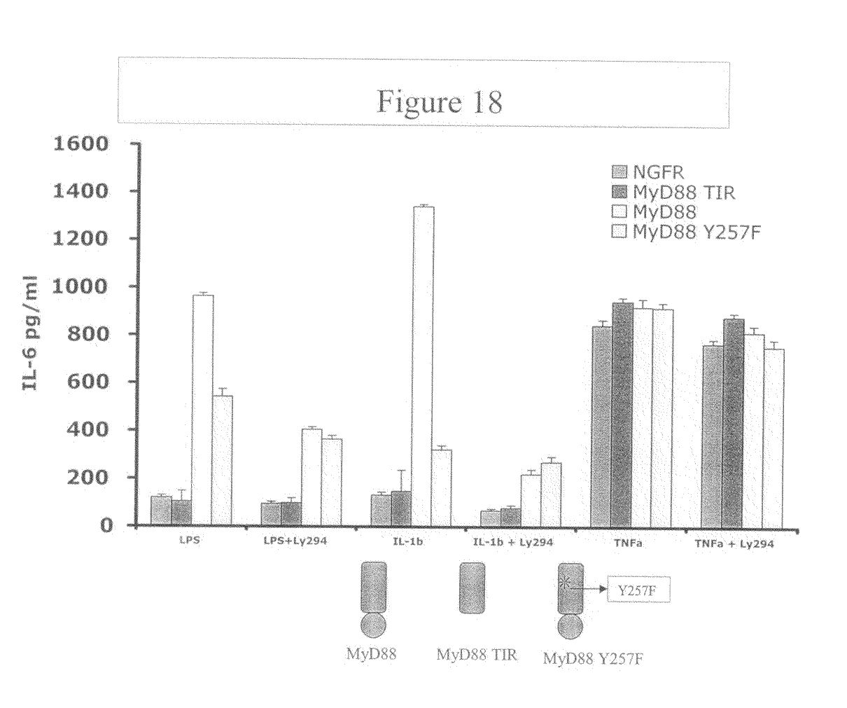

The following experiments were designed to determine the role of MyD88 in T cells with respect to the potential PI3K binding site in MyD88 following activation of a TLR on T cells. FIG. 17B depicts appropriate mutants for expression in MyD88-deficient T cells to determine the role of MyD88 with respect to the potential PI3K binding site in MyD88. It was observed that optimal IL-6 responses to LPS or IL-1 is dependent on the Y257 residue in a putative SH2 binding sequence present in the MyD88 TIR domain (FIG. 18).

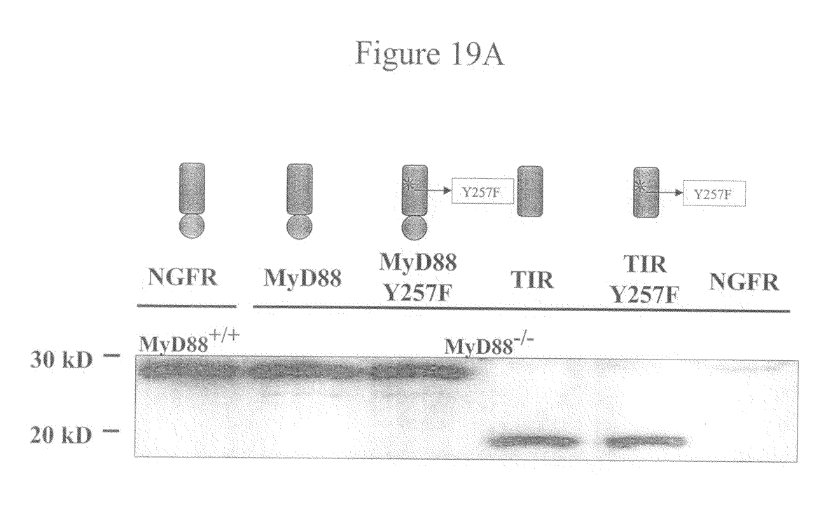

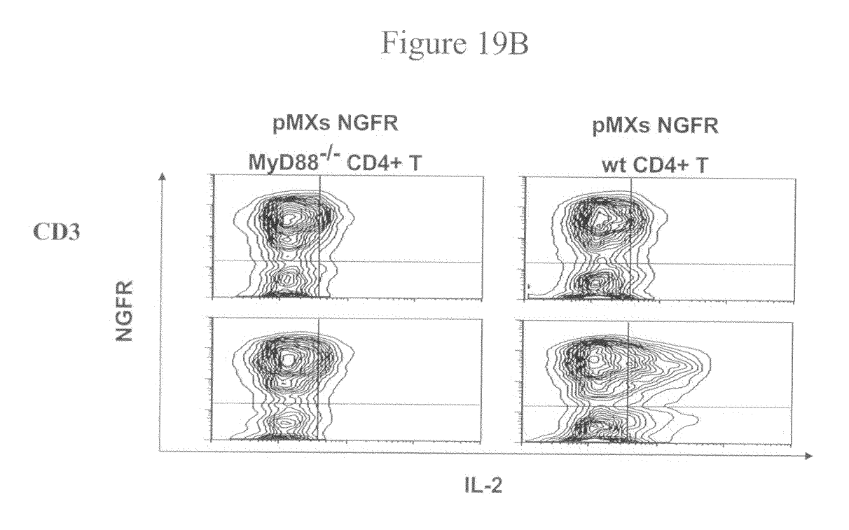

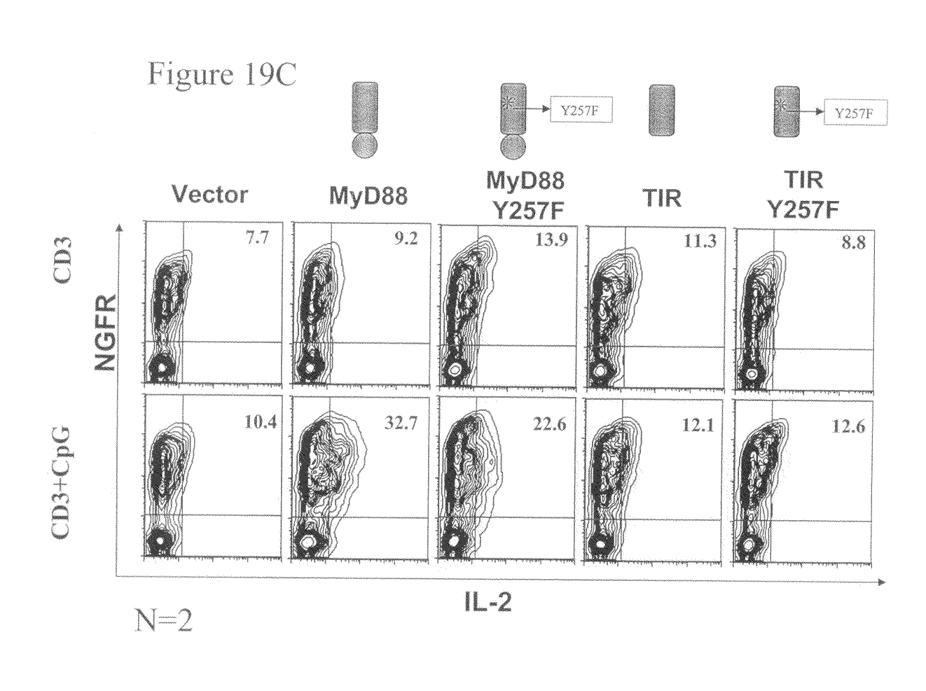

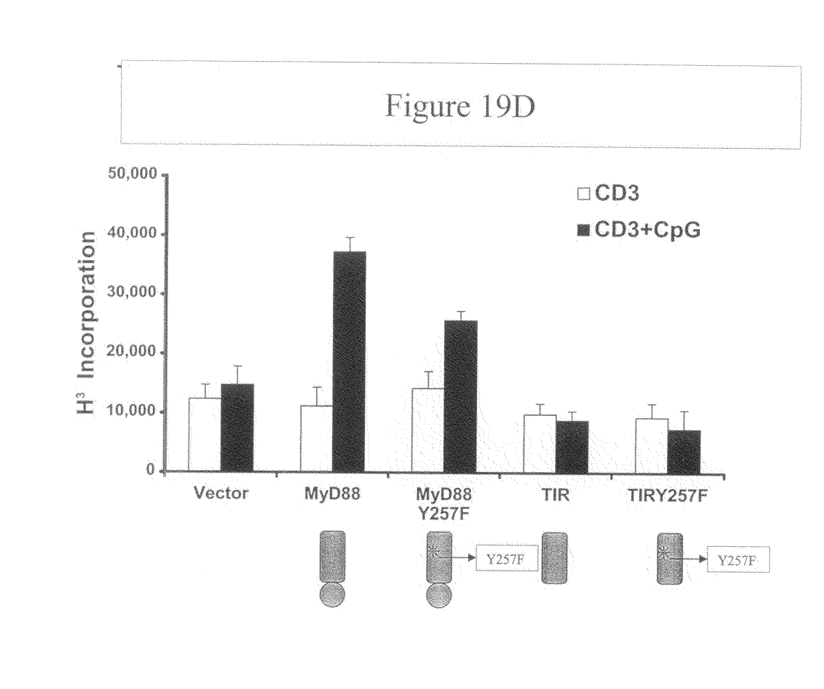

The next set of experiments was designed to assess the effects of CpG ODN costimulatory activity in CD4+ T cells. The results demonstrate that MyD88 is required to activate downstream targets of PI3-kinase such as Akt and GSKα for optimal CpG ODN induced proliferation of CD4+ cells and IL-2 synthesis (FIGS. 19A-19D).

### Example 5

### Foxp3 Expression in Natural Tregs

The forkhead transcription factor, Foxp3, encoded by the FOXP3 gene, is a marker of regulatory T cells (Tregs). It is known that Foxp3 expression, and thus regulatory function, can be induced under certain circumstances in Foxp3-negative T cells by exposure to TGF-b. It has not been established in the art whether Foxp3 expression can be abrogated in pre-existing Foxp3⁺ T cells.

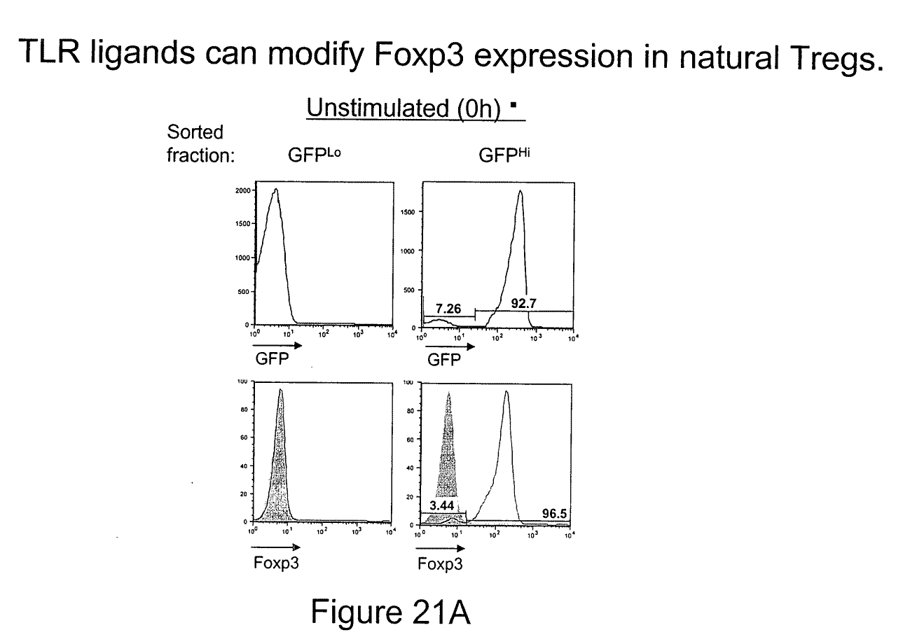

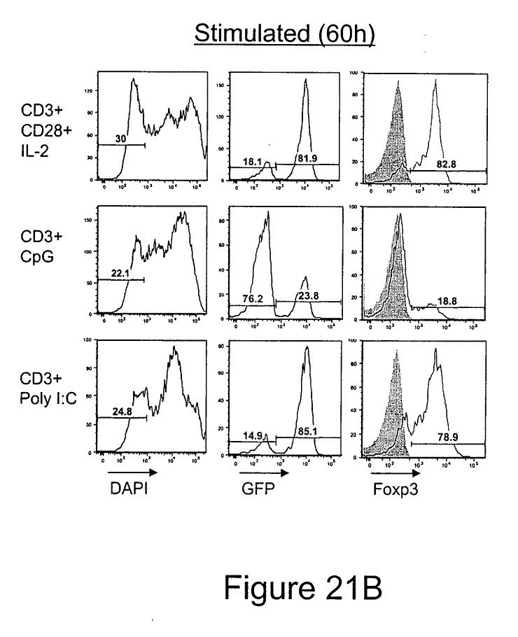

In order to study the effect of stimulation conditions on Foxp3 expression, T cells were from mice having a reporter construct introduced into the FOXP3 locus (Betteli et al., 2006, Nature 441:235-238) by sorting cells based on the GFP-reporter construct. The reporter construct expresses both Fox3p and the fluorescent protein GFP. T cells that are Foxp3⁻ (i.e. GFP⁻) and T cells that are Foxp3⁺ (i.e. GFP⁺) were isolated (FIG. 21A). Foxp3 expression was then assessed under different stimulation conditions,

As shown in FIG. 21B, Foxp3⁺GFP⁺ cells (equivalent to natural Tregs) maintained Foxp3 expression when activated by anti-CD3 and anti-CD28 antibodies plus IL-2 or when activated by anti-CD3 plus the TLR3 ligand, poly I:C. However, the TLR9 ligand, CpG, induced a loss of Foxp3 expression.

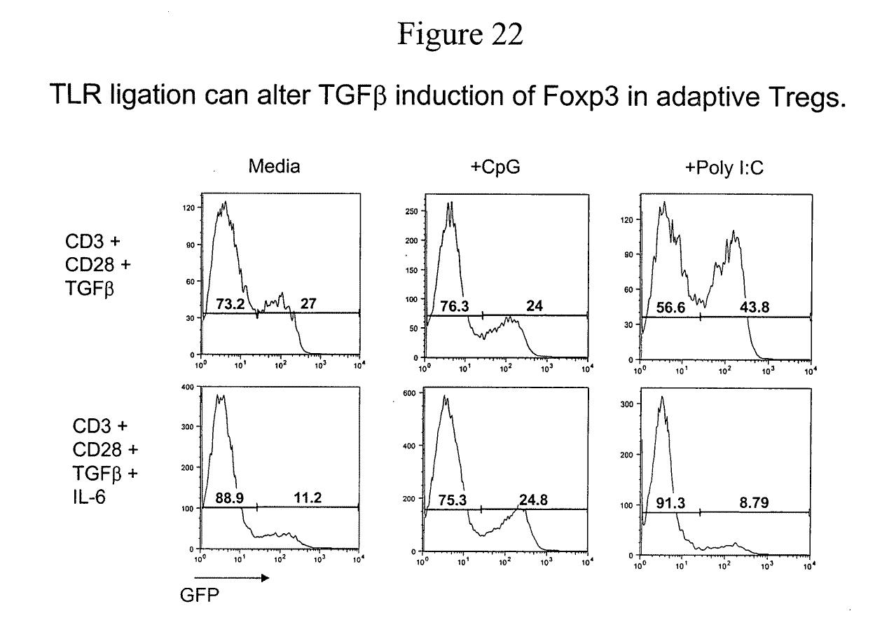

As shown in FIG. 22 (top three panels), and as expected, stimulation of Foxp3⁻GFP⁻ cells (equivalent to non-regulatory T cells) with anti-CD3 and anti-CD28 antibodies and transforming growth factor beta (TGF-b) promoted Foxp3 expression. Unexpectedly, this effect was augmented by the addition of poly I:C. The effect was not, however, augmented by CpG. The addition of IL-6 to the stimulation conditions blocked the induction of Foxp3 by TGF-b (FIG. 22, bottom three panels).

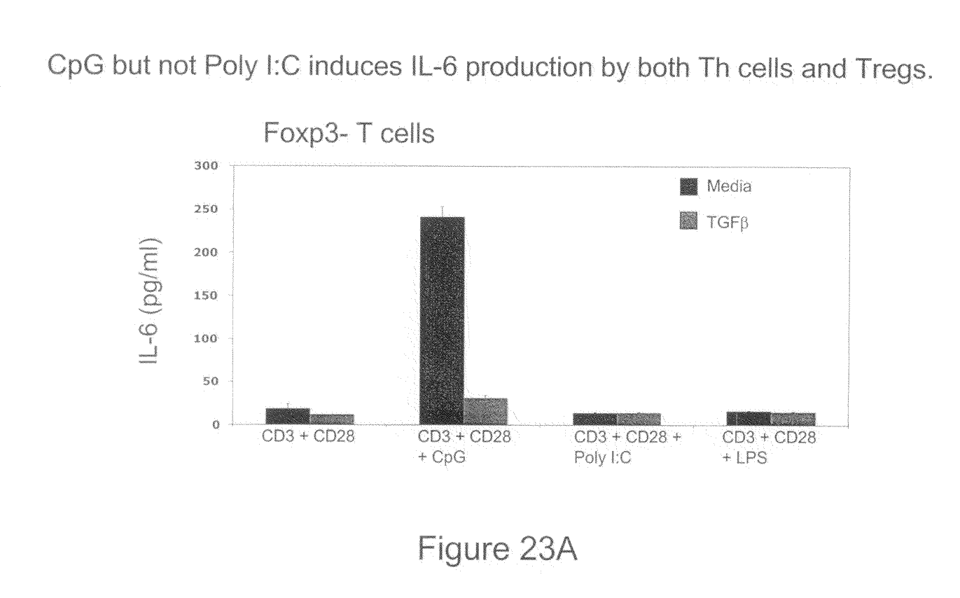

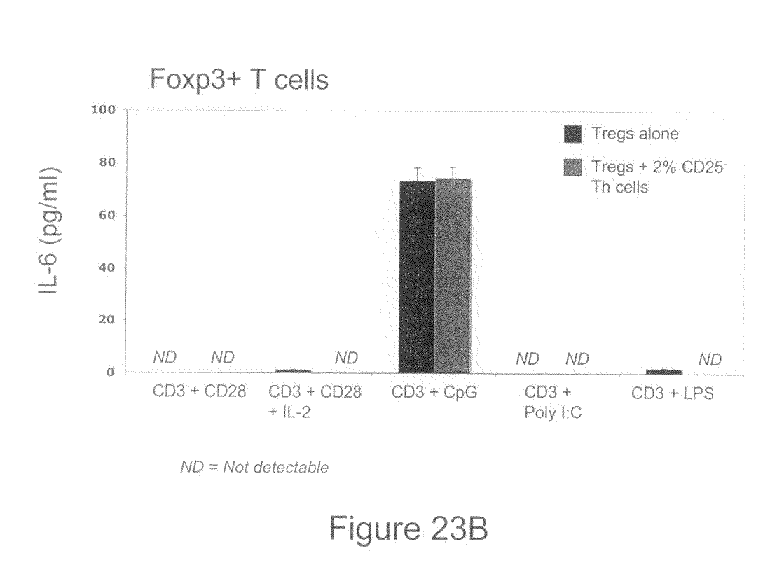

Unexpectedly, CpG induced IL-6 production in both stimulated Foxp3⁻ T cells and stimulated Foxp3⁺ T cells (FIGS. 23A and 23b). This is the first demonstration of induction of IL-6 in T cells by TLR ligands. While not wishing to be bound by theory, it is possible that the induction of IL-6 by CpG may explain the lack of induction of Foxp3 expression by CpG observed in FIG. 21, since IL-6 blocks induction of Foxp3 expression by TGF-b (FIG. 22).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition for increasing T cell proliferation and cytokine production, the composition comprising: a Toll-like receptor (TLR) ligand wherein said TLR ligand is capable of activating a TLR on a T cell; and a T cell stimulator wherein said stimulator is capable of activating said T cell.

2. The composition of claim 1, wherein said TLR ligand is capable of activating TLR9.

3. The composition of claim 1, wherein said TLR ligand is capable of activating TLR3.

4. The composition of claim 1, wherein said TLR ligand is selected from the group consisting of CpG DNA and poly I:C.

5. The composition of claim 1, wherein said TLR ligand is a combination of CpG DNA and poly I:C.

6. The composition of claim 1, wherein said T cell stimulator comprises an antibody selected from the group consisting of an anti-CD3 antibody and an anti-CD28 antibody.

7. The composition of claim 1, wherein said T cell stimulator comprises both an anti-CD3 antibody and an anti-CD28 antibody.

8. The composition of claim 1, further comprising an antigen having at least one epitope, wherein said epitope is capable of eliciting an immune response in a mammal.

9. The composition of claim 1, further comprising a T cell.

10. The composition of claim 9, wherein said T cell is an activated T cell.

11. The composition of claim 10, wherein said activated T cell exhibits an enhanced survival characteristic.

12. The composition of claim 1, wherein said cytokine is selected from the group consisting of IL-2 and IL-6.

13. A composition for increasing T cell proliferation and cytokine production, the composition comprising a T cell stimulator and one of CpG and poly I:C, wherein said stimulator is capable of activating said T cell.

14. The composition of claim 13, wherein said T cell stimulator comprises an antibody selected from the group consisting of an anti-CD3 antibody and an anti-CD28 antibody.

15. The composition of claim 13, wherein said T cell stimulator comprises both an anti-CD3 antibody and an anti-CD28 antibody.

16. A T cell that is genetically modified to express elevated levels TLR3 and/or TLR9 compared to an otherwise identical T cell not so modified, wherein contact of TLR3 and/or TLR9 with a TLR ligand enhances the survival of said genetically modified T cell.

17. The T cell of claim 16, wherein said cell exhibits an enhanced survival characteristic compared to an otherwise identical T cell not so modified.

18. The cell of claim 16, wherein said cell is capable of regulating an immune response.

19. The cell of claim 18, wherein said immune response is associated with a disease selected from the group consisting of an infectious disease, a cancer, and an autoimmune disease.

20. A method of inducing T cell proliferation and promoting cytokine production, the method comprising activating a T cell with a composition comprising a Toll-like receptor (TLR) ligand wherein said TLR ligand is capable of activating a TLR on said T cell; and a T cell stimulator wherein said stimulator is capable of activating said T cell.

21. The method of claim 20, wherein said T cell proliferation is dependent on NF-κB.

22. The method of claim 20, wherein said TLR ligand is capable of activating TLR9.

23. The method of claim 20, wherein said TLR ligand is capable of activating TLR3.

24. The method of claim 20, wherein said TLR ligand is selected from the group consisting of CpG DNA and poly I:C.

25. The method of claim 20, wherein said TLR ligand is a combination of CpG DNA and poly I:C.

26. The method of claim 20, wherein said T cell stimulator comprises an antibody selected from the group consisting of an anti-CD3 antibody and an anti-CD28 antibody.

27. The method of claim 20, wherein said T cell stimulator comprises both an anti-CD3 antibody and an anti-CD28 antibody.

28. The method of claim 20, wherein the proliferation of said T cell is independent of the presence of an antigen presenting cell.

29. The method of claim 20, wherein said cytokine is selected from the group consisting of IL-2 and IL-6.

30. A method of inducing T cell proliferation and promoting cytokine production, the method comprising activating a T cell with a composition comprising a T cell stimulator and one of CpG and poly I:C, wherein said stimulator is capable of activating said T cell.

31. The method of claim 30, wherein said T cell stimulator comprises an antibody selected from the group consisting of an anti-CD3 antibody and an anti-CD28 antibody.

32. The method of claim 30, wherein said T cell stimulator comprises both an anti-CD3 antibody and an anti-CD28 antibody.

33. The method of claim 30, wherein the proliferation of said T cell is independent of the presence of an antigen presenting cell.

34. The method of claim 30, wherein said cytokine is selected from the group consisting of IL-2 and IL-6.

35. A method of enhancing an immune response in a mammal, the method comprising administering to said mammal a composition comprising: a Toll-like receptor (TLR) ligand wherein said TLR ligand is able to activate a TLR on said T cell; and a T cell stimulator wherein said stimulator is able to activate said T cell.

36. A method of enhancing an immune response in a mammal, the method comprising administering to said mammal a composition comprising a T cell stimulator and one of CpG and poly I:C, wherein said stimulator is able to activate said T cell.

37. A method of enhancing an immune response in a mammal, the method comprising administering to said mammal a T cell, wherein said T cell has been stimulated with a composition comprising: a Toll-like receptor (TLR) ligand wherein said TLR ligand is able to activate a TLR on said T cell; and a T cell stimulator wherein said stimulator is able to activate said T cell.

38. A method of enhancing an immune response in a mammal, the method comprising administering to said mammal a T cell, wherein said T cell has been stimulated with a composition comprising a T cell stimulator and one of CpG and poly I:C, wherein said stimulator is able to activate said T cell.

39. A method of suppressing an immune response in a mammal, the method comprising administering to said mammal a composition that inhibits and/or reduces expression of a TLR and/or a downstream signaling molecule thereof in a T cell in said mammal, wherein said composition is selected from the group consisting of a small interfering RNA (siRNA), an antisense nucleic acid and a ribozyme.

40. A method of suppressing an immune response in a mammal, the method comprising administering to said mammal a composition that inhibits and/or reduces activity of a TLR and/or a downstream signaling molecule thereof in a T cell in said mammal, wherein said composition is selected from the group consisting of a transdominant negative mutant, an intracellular antibody, a peptide and a small molecule.

41. A method for modulating Foxp3 expression in a T cell, the method comprising activating a T cell with a composition comprising a T cell stimulator and one of CpG and poly I:C, wherein said stimulator is capable of activating said T cell.

42. The method of claim 41, wherein the composition comprises CpG and Foxp3 expression is reduced.

43. The method of claim 41, wherein the composition comprises poly I:C and Foxp3 expression is induced.

44. The method of claim 43, wherein said composition further comprises transforming growth factor beta (TGF-b).
